(19) United States
(12) Patent Application Publication
(10) Pub. No.: US 2003/0124551 A1
(43) Pub. Date: Jul. 3, 2003

(54) Agents implicated in endometriosis

(76) Inventors: Helen Pappa, Wokingham (GB); Mirna Lnenicek-Allen, Wokingham (GB)

Correspondence Address: KLAUBER & JACKSON, 411 HACKENSACK AVENUE, HACKENSACK, NJ, 07601

(21) Appl. No.: 10/139,604

(22) Filed: May 3, 2002

Related U.S. Application Data
(63) Continuation of application No. PCT/GB00/04228, filed on Nov. 3, 2000.

(30) Foreign Application Priority Data
Nov. 3, 1999 (GB) 9926081.2
Nov. 3, 1999 (GB) 9926074.7
Nov. 3, 1999 (GB) 9926079.6
Nov. 3, 1999 (GB) 9926076.2

(51) Int. Cl. C12Q 1/68; C12P 19/34

(52) U.S. Cl. 435/6; 435/91.2

(57) ABSTRACT

The present invention relates to the discovery of genes and their products that are associated with the disease endometriosis. It has been discovered that cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) and podocalyxin are all implicated in this disease. The discovery of these associations has clear implications for the diagnosis and treatment of endometriosis and related conditions.

The present invention relates to the discovery of genes and their products that are associated with the disease endometriosis. The discovery of these agents has implications for the diagnosis and treatment of endometriosis and related conditions.

Endometriosis is the name given to the disease resulting from the presence of endometrial cells outside of the uterine cavity. This disease affects women during their childbearing years with deleterious social, sexual and reproductive consequences. Endometriosis has been proposed as one of the most commonly-encountered diseases of gynaecology, with the incidence of endometriosis in the general population being estimated to be around 5%, although it is thought that at least 25% of women in their thirties and forties may be suffering from this disease.

The development and maintenance of endometriosis involves the establishment and subsequent sustained growth of endometrial cells at ectopic sites, most commonly the pelvic peritoneum and ovaries, following retrograde menstruation (see Thomas & Prentice (1992) Repro. Med. Rev. (1): 21-36). Implantation of autologous non-malignant ectopic tissue is a unique phenomenon suggesting that an abnormal host response may be present in women who develop this disease. This theory is supported by the fact that only a minority of women will develop the disease in spite of the common occurrence of retrograde menstruation as a source of endometrial tissue.

There are many theories proposed for the origin of endometriosis and various cellular and biochemical constituents of the peritoneal fluid have been reported to play an important role in the pathogenesis of this disease. Alterations in multiple aspects of both humoral immunity and cell-mediated immunity have also been reported in suffering individuals.

The heritable aspects of endometriosis have been investigated in several studies (Moen & Magnus (1993) Acta Obstet. Gynecol. Scand., 72: 560-564; Kennedy et al, (1995) J. Assist. Repro. Gen., 12(1): 32-35; Malinak et al (1986) Am. J. Obstet. Gynecol., 137(3): 332-337; Treloar et al., (1999) Fertility Sterility 71(4) 701-710). On the basis of these studies, it has been hypothesised that endometriosis has a genetic basis. However, the precise aetiology of this disease still remains unknown.

The growth and development of endometrial tissue appears to depend on the presence of oestrogen. Drugs have thus been developed that reduce the body's oestrogen content in order to reduce the growth of endometrial implants at ectopic sites. Strategies include mimicking pregnancy, preventing ovulation using contraceptive agents, blocking the action of progesterone and mimicking the menopause. Although some of these drugs have proved successful, many cause unpleasant side-effects including infertility, which mean that treatment must be discontinued to avoid the side-effects becoming permanent. Furthermore, all drugs described to date act by relieving the symptoms of the disease and are not in any sense curative. This makes a patient permanently dependent on the drug if the symptoms of disease are to be kept at bay.

Presently, the only treatment of endometriosis that is effective in the long term involves surgery. Certain developments in the treatment of endometriosis, such as the identification of vascular endothelial growth factor (VEGF) which possesses angiogenic activity and which is thought to be partly responsible for the establishment and development of endometriosis, have in part paved the way for the development of therapeutic agents that are effective in treating the disease and/or preventing its incidence. However, there remains a great need for the discovery of agents with effective prophylactic, therapeutic and diagnostic value against endometriosis.

The discovery of factors that determine susceptibility to this disease would allow medical intervention at an early stage of the disease before the more severe symptoms manifest themselves. Such a discovery would aid the treatment of infertility and facilitate the management of the chronic pain that this disease causes.

Furthermore, the early stages of this condition are essentially asymptomatic, meaning that most diseased individuals do not present in the clinic until the disease is quite well advanced. Even at this stage, diagnosis of the disease relies on invasive measures such as laparoscopy. Treatment is also difficult and expensive, normally involving surgery.

There thus remains a great need for the identification of factors in the body that are involved in the development and progression of endometriosis. The discovery of such agents would allow the development of diagnostic agents that would allow the disease to be diagnosed and, if necessary, treated at an early stage. Identification of the genes and proteins implicated in this disease would also allow the development of therapeutically-effective compounds, for example, using methods of high-throughput screening to identify small molecules of interest.

In order to elucidate the molecular events that lead to or that progress endometriosis, the inventors have monitored the patterns of expression between “diseased” (ectopic) and “healthy” (eutopic) states in tissues associated with endometriosis.

### SUMMARY OF THE INVENTION

According to the present invention there is provided a method of screening for a gene that is associated with endometriosis comprising comparing the pattern of gene expression in a diseased endometrium tissue from a patient suffering from endometriosis to the pattern of gene expression in healthy endometrium tissue from the same patient suffering from endometriosis, and selecting a gene whose level of expression differs between healthy and diseased tissues.

It has been discovered, surprisingly, that by comparing levels of gene expression in healthy and diseased tissues that are derived from the same patient, it is possible to identify genes whose aberrant expression is associated with endometriosis. To our knowledge, this is the first study that has evaluated the role played by differential gene expression in endometriosis. The inventors have in this manner proven that endometriosis has a genetic basis.

By “diseased endometrium tissue” is meant any ectopic endometrial tissue. This tissue type may be in any anatomical location, and generally is to be found in the region of the ovary, peritoneum, or recto-vagina. However, it should be noted that ectopic endometrium has been found in locations as disparate as the brain and lung. Diseased endometrium tissue may be determined histologically using markers or by looking for endometrial glands and stromal elements in tissue at ectopic sites.

By “healthy endometrium tissue” is meant endometrial tissue present within the uterine cavity (i.e. eutopic endometrium) that is not diseased.

By “pattern of gene expression” is meant the composition of genes that are expressed within the cells that make up a tissue of interest. This term is intended to refer both to the level at which the gene is expressed and to the distribution of gene expression within this tissue. The pattern of gene expression thus reflects the composition of proteins that are expressed in the tissue cells. One strength of the method of the present invention lies in its ability to assess gene expression, since it is gene expression levels that are ultimately causative of disease. Of course, the pattern of gene expression does not correspond exactly to the pattern of protein expression, since the translation of MRNA transcribed from each gene is regulated by complex protein-dependent systems. The inventors consider that measuring aberrant protein levels may be misleading, since such aberrant levels of protein will in some cases be the result of an upstream gene processing event.

The inventors have found that it is important to compare diseased and non-diseased tissues from the same patient in order that the differences in gene expression found may be linked directly to the endometriosis condition. A method comparing tissues from different patients would be prone to giving false positives, since any differential expression patterns might depend on innate differences in the patterns of expression between different individuals (for example, resulting from one or more polymorphisms within a gene sequence) rather than reflecting differences that are indicative or causative of the endometriosis condition.

In some instances, it has been found that the expression of various genes implicated in endometriosis varies throughout the menstrual cycle. Accordingly, levels in diseased tissue will not necessarily be aberrant for the entire menstrual cycle, but may only be altered for part of the cycle. In some instances, the variation in the expression of a gene is in fact the exact opposite to that found in healthy tissue. For example, a gene whose expression is normally high at the early stage of the cycle, and which is lowest at the middle of the cycle, becomes highest at the middle of the cycle and low at the beginning of the cycle. The inventors have thus found that in many cases it is important to compare tissues at the same stage in the menstrual cycle.

The method according to the invention may utilise any method of monitoring the differential expression of genes. Examples include indexing differential display reverse transcriptase polymerase chain reaction (DDRT-PCR; Mahadeva et al. (1998) J. Mol.Biol. 284, 1391-1398; International patent application WO94/01582), subtractive mRNA hybridisation (see “Advanced Molecular Biology”, R. M. Twyman (1998) Bios Scientific Publishers, Oxford, p336; “Nucleic Acid Hybridization”, M. L. M. Anderson (1999) Bios Scientific Publishers, Oxford, pp199-202; Sagerstrom et al. (1997) Annu. Rev. Biochem. 66: 751-783), the use of nucleic acid arrays or rmicroarrays (see Nature Genetics, (1999), vol 21 suppl; 1-61), and the serial analysis of gene expression (SAGE; Velculescu et al., Science (1995) 270; 484-487).

The inventors have used the DDRT-PCR technique, a highly consistent and reproducible method that in this instance has allowed the identification of genes that are differentially expressed in endometriotic tissues. Some genes appear to be expressed at lower levels than normal, whilst some are overexpressed relative to the levels in healthy tissue.

The indexing DDRT-PCR technique used by the inventors is highly reproducible and results in a low level of false positives. This is attributable to the fidelity of T₄ DNA ligase-catalysed adapter ligation, in contrast to the stochastic nature of PCR amplification based upon the low stringency annealing of arbitrary primers that is associated with the differential display and arbitrarily-primed PCR protocols conventionally used in the art.

In the DDRT-PCR technique, digestion of a cDNA population with a Type II S restriction endonuclease produces fragments with every combination of possible bases in the cohesive ends (see FIG. 1). Under stringent conditions, the specific ligation of adapters with perfectly complementary overhangs partitions the cDNA fragments into non-overlapping sub-populations. Internal cDNA restriction fragments are exponentially amplified by adapter primer PCR and visualised by non-denaturing polyacrylamide gel electrophoresis (see FIG. 2). Comparisons of patterns that are produced from diseased and non-diseased cDNA populations thus provide details of differentially-expressed gene fragments.

Mechanisms by which the aberrant expression of a gene may be manifested as the disease endometriosis are, of course, varied, as will be clear to those of skill in the art. The method of the invention identifies any variation in gene expression whatsoever; the variation may be the result of a mutation or polymorphism in the sequence of the gene whose expression is aberrant. As used herein, the term “mutation” is intended to include mutations such as deletions, microdeletions, aneuploidies, translocations, inversions, insertions, multiple repeats, and multiple and single-base nucleotide substitutions.

The presence of a mutation may result in either a decreased or an increased amount of gene transcription, for example, through altering the activity of a promoter or enhancer that governs regulation of the gene, by altering mRNA stability or by altering the efficiency of transcription termination. Variation in gene expression may also result from aberrant regulation of the gene, for example, as a result of altered levels or activity of a regulatory protein. Again, typical mechanisms include altered promoter or enhancer activity, such as by affecting the binding of a regulatory protein to the promoter or enhancer site, and altered mRNA stability.

Generally, the aberrant expression of a gene is manifested physiologically through alterations in the biological activity of the protein encoded by the gene (the gene product). By “biological activity” is meant the activity of the wild type gene product and may comprise an enzymatic activity, a binding activity, a structural activity or a co-factor activity, by participating in a biochemical reaction in cells. By “wild type” is meant the phenotype that occurs naturally in the majority of the population of the species and which is manifested in healthy tissue.

The biological activity of the gene product may be either increased, decreased or regulated abnormally in diseased tissue relative to its activity in healthy tissue. The altered biological activity of gene products in diseased tissues produced from aberrant expression of genes, or as a result of a gene mutation, will typically be due to changes in the amounts of gene product expressed in the diseased tissue. The resulting physiological imbalance, referred to herein as an abnormal stoichiometry, then leads either directly or indirectly to the symptoms of the disease.

In some instances, the altered biological activity of the gene product may lead directly to the symptoms of the endometriosis disease. For example, the gene product may be a structural protein or a metabolic enzyme that when over- or under-expressed in endometrium tissue causes a local disruption that is manifested as endometriosis disease. If a gene product is overexpressed, an inhibitor (including antagonists) can be used to reduce the biological activity to the normal levels; if the gene product is underexpressed, an activator (including agonists) can be used to elevate its biological activity.

In other cases, the activity of the gene product has downstream effects on other proteins that are themselves causative of disease, so that the gene product indirectly causes endometriosis symptoms. Such downstream effects might be caused by a direct interaction of the gene product with one or more other proteins. In this way, the correct physiological balance that is reflected in healthy endometrial tissue, may be upset.

Downstream effects might in other instances be caused by affecting the expression of genes encoding proteins that are themselves directly causative of disease. Similar mechanisms are reflected in a variety of diseases affecting humans and other mammals and will be appreciated by those of skill in the art.

Examples of proteins implicated in endometriosis by the method of the first aspect of the invention include cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) and podocalyxin.

### DIAGNOSIS

According to a further aspect of the present invention, there is provided a method for detecting or diagnosing endometriosis in a patient, comprising assessing the level of expression of a gene or gene product selected by the method of the first aspect of the invention in tissue from said patient and comparing said level of expression to a control level of expression, wherein a level of expression that is different to said control level is indicative of endometriosis.

This aspect of the present invention provides therefore methods for diagnosing individuals as suffering from, or as being likely to suffer from, endometriosis.

In one respect, the methods of this aspect of the invention provide useful diagnostic tools for predicting the potential susceptibility of an individual to endometriosis. The asymptomatic nature of this disease during its early stages makes this tool particularly useful, since a diagnosis may be made at an early enough stage in the disease to curb its progression or further development.

One other aspect of this disease that makes a diagnostic tool so valuable is the fact that many women do not develop symptoms of endometriosis until well into their thirties or forties. This means that the disease is well-advanced by the time medical intervention becomes possible. The screening of large populations of individuals susceptible to endometriosis would allow prophylactic measures to be taken in the clinic.

If large-scale screening of the general population is not possible, for example for reasons of economy, it may be that asymptomatic individuals from families having a history of endometriosis can be selectively screened using the methods of this aspect of the invention, so allowing for diagnosis prior to the appearance of any symptoms of the disease. Susceptible individuals may then take appropriate precautions, as necessary.

Another aspect of the invention provides a method of monitoring the therapeutic treatment of endometriosis in a patient, comprising monitoring over a period of time the level of expression of a gene implicated in endometriosis in said patient and comparing said level of expression to a control. Lowering or raising this level of expression in a diseased tissue or body fluid over a period of therapeutic treatment towards the control level may be indicative of regression of said disease.

Of course, in all the diagnostic methods discussed above, a combination of genes, gene products, agonists and/or antagonists may be used, if appropriate. Such an approach will reduce the number of false positives identified by the screen, so reducing the need for expensive confirmatory techniques that will usually necessitate the intervention of a clinician. A combined approach may in certain circumstances also reduce the number of false negatives, allowing a clinician greater confidence in the screen being used.

Diagnosis of Gene Expression Levels

Diagnosis may be by monitoring gene expression itself, or by monitoring levels of gene product expressed from the gene whose aberrant expression is associated with endometriosis.

Suitable tissues for biopsy include body fluids such as blood, peritoneal fluid, urine and saliva, and solid tissues such as endometrium, obtained from the patient for diagnosis. Preferably, biopsy tissue is taken from ectopic endometrium, or any other affected area (ovaries, peritoneal cavity, rectovagina) that is potentially diseased and suitable for diagnosis. The level of gene expression in this ‘diseased’ tissue is compared to a control level, such as the level found in healthy endometrium tissue obtained from the patient, or a standard control. In some instances the level of gene expression in the eutopic endometrium might be an indicator of endometriosis if this gene is expressed in patients of endometriosis and is completely absent in healthy individuals.

Any suitable technique that allows for the quantitative assessment of the level of expression of a specific gene in a tissue may be used. Comparison may be made by reference to a standard control, or to a control level that is found in healthy tissue. For example, levels of a transcribed gene can be determined by Northern blotting, or/and RT-PCR. With the advent of quantitative (real-time) PCR, the number of gene copies present in any RNA population can accurately be determined by using appropriate primers for the gene of interest. Levels of a transcribed gene or/and an entire gene family and pathway can be now monitored by hybridisation on gene arrays that contain nucleic acid sequences from all the genes of interest, immobilised on a solid surface.

Susceptibility to endometriosis may be assessed by contacting nucleic acid isolated from patient tissue samples with a nucleic acid probe under stringent conditions that allow the formation of a hybrid complex between the nucleic acid probe and the gene implicated in endometriosis and detecting the presence of a hybrid complex in the samples. For use as a diagnostic agent, it may be preferable to label the nucleic acid probe to aid its detection. This level of detection is compared to control levels, such as, for example, gene levels from a healthy specimen or a standard control; detection of altered levels of the hybrid complex from the patient tissue is indicative of endometriosis. Techniques discussed above, such as antisense technology, may be used to assess gene expression levels in this manner; the degree of specific binding of oligonucleotide may be assessed as an indication of the level of gene expression.

In order to detect nucleic acid in patient tissue, a number of techniques may be used, as will be clear to those of skill in the art. Preferably, the nucleic acid is amplified specifically using polymerase chain reaction technology (PCR). The nucleic acid may also be labelled and hybridised on a gene array, in which case the gene concentration will be directly proportional to the intensity of the radioactive or fluorescent signal generated in the array.

Firstly, the nucleic acid must be separated from patient tissue for testing. Suitable methods will be known to those of skill in the art. For example, RNA may be isolated from the tissue to be analysed using conventional procedures (see, for example, MacDonald et al., (1987) Meth Enzymol 152: 219), such as are supplied by QIAGEN technology. This RNA is then reverse-transcribed into DNA using reverse transcriptase and the DNA molecule of interest may then be amplified by PCR techniques using specific primers. As used herein, amplification of nucleic acid sequences is intended to include any method or technique that is capable of increasing the amount of a specific nucleic acid molecule. A preferred amplification technique is PCR. More preferably, particularly in those cases where more than one spliced variant of the gene exists, a nested PCR reaction may be more informative.

Diagnostic procedures may also be performed directly upon patient tissue obtained, for example, from biopsies. Hybridisation or amplification assays, such as, for example, Southern or Northern blot analysis, in situ hybridisation analysis, immunohistochemistry, single-stranded conformational polymorphism analysis (SSCP) and PCR analyses are among techniques that are useful in this respect. If desired, target or probe nucleic acid may be immobilised to a solid support such as a microtitre plate, membrane, polystyrene bead, glass slide or other solid phase.

As the skilled reader will appreciate, the emerging field of nucleic acid arrays is generating a large number of powerful tools for the study of DNA and RNA variation. These methods, based on techniques pioneered by Schena et al., 1995 (Science 270: 467-470) and Fodor et al., 1991 (Science 251, 767-773) facilitate the evaluation of variations in the nucleic acid sequence of DNA or RNA samples and so allow the identification and genotyping of mutations and polymorphisms in these sequences. Recent advances in this technology include those reported by Brown and Botstein (1999, Nature Genet 21:25-32); Hacia (1999, Nature Genet 21, 42-27) and Wang et al., (1998, Science 280:1077-1082) and are reviewed generally in Nature Genetics 21, supplement 1 (January 1999). Many of these techniques are applicable to the present invention, including improvements in microarray technology that will undoubtedly be developed over the coming years. Gene arrays containing certain pathways (Clontech; Atlas Select Human Tumor Arrays) and gene families (Clontech, Atlas Select Human Tumor Arrays, R&D Systems' Cytokine Expression Array) are becoming commercially available even now.

Recently, the detection of differential gene expression in patient samples has been demonstrated using nucleic acid arrays such as those described above. Hybridisation of RNA or DNA to DNA chips allows monitoring expression of mRNAs or the occurrence of polymorphisms in genomic DNA in a high-throughput manner.

Two array formats are currently commercially available; filter arrays containing cDNA sequences, and glass chips containing cDNA or in situ synthesized oligonucleotide sequences. The sensitivity of filtered arrays is reported to be limited to high- and medium-abundance genes while DNA chips (glass) can detect low abundance genes.

Comparison of expression between two samples (healthy, diseased) on filter arrays may be performed by comparing healthy and diseased RNA samples to separate duplicate filters. Alternatively, a single filter may be used that must be stripped and hybridized sequentially.

Direct comparison of gene expression in two samples can be achieved on glass arrays by labelling the two samples with different fluorophores. This technique allows the evaluation of repression of gene expression as well as induction of expression. The two fluorescently-labeled cDNAs are then mixed and hybridised on a single slide array. Glass arrays have the advantage of allowing the simultaneous analysis of two samples on the same array under the same hybridisation conditions. However, these methods are very sample intensive requiring up to 100 μg of RNA sample for a fluorescent probe (50 ng of total RNA is required for a single experiment with nylon arrays).

Gene arrays containing sequences of genes implicated in endometriosis will allow high-throughput screening of individuals for diagnostic purposes or tailor-made treatments. Additionally, such arrays may allow evaluation of the success or failure of a drug treatment in the event that induced gene products are useful as surrogate markers. In support of this possibility, a recent report describes the phenotypic diversity of breast tumours, captured using cDNA microarrays. These arrays provided a distinctive molecular portrait of each tumour and allowed the classification of the tumours into subtypes distinguished by their differences in their gene expression patterns (see Peron C. M. et al., 2000, Nature 406, 747-752).

Arrays of polynucleotides whose sequences correspond to, or are complementary to the sequences of genes identified by the method according to the first aspect of the invention therefore form a further aspect of the invention. Such an array should include at least two nucleic acid molecules, wherein each of said nucleic acid molecules either corresponds to the sequence of, is complementary to the sequence of, or hybridises specifically to one of the genes herein implicated in endometriosis. As stated above, suitable genes include cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1 and podocalyxin.

Nucleic acid molecules for the detection of multiple gene types may be included on arrays according to this aspect of the invention. Such an array may contain nucleic acid molecules that either correspond to the sequence of, are complementary to the sequence of, or hybridise specifically under high stringency conditions to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or34 or more of the genes implicated in endometriosis by the method of the first aspect of the invention. The term “hybridization” as used here refers to the association of two nucleic acid molecules with one another by hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. Molecular Cloning; A Laboratory Manual, Second Edition (1989)). In accordance with these principles, the inhibition of hybridization of a complementary molecule to a target molecule may be examined using a hybridization assay; a substantially homologous molecule possessing a greater degree of homology will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511). “Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate, pH8.0), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization in the methods of this aspect of the invention are those of high stringency.

Preferably, arrays according to the invention contain nucleic acid molecules which consist of between twelve and fifty nucleotides, more preferably, between fifteen and thirty-five nucleotides.

Protein arrays form a further aspect of the invention, that are useful for the diagnosis of endometriosis and also for the identification of additional molecules that are implicated in this disease. Recent developments in the field of protein and antibody arrays allow the simultaneous detection of a large number of proteins. Low-density protein arrays on filter membranes, such as the universal protein array system (Ge H, (2000) Nucleic Acids Res. 28(2), e3) allow imaging of arrayed antigens using standard ELISA techniques and a scanning charge-coupled device (CCD) detector on an optically flat glass plate containing 96 wells. Immuno-sensor arrays have also been developed that enable the simultaneous detection of clinical analytes.

A number of methods for fabricating antibody arrays have been reported by Dolores J. Cahill in Proteomics: A Trends Guide, Trends in Biotechnology, July 2000, pg47-51. The advantage of these technologies is the extensive parallel analysis of several proteins implicated in disease simultaneously. Additionally, by using protein arrays, protein expression can be profiled in bodily fluids, such as in sera of healthy or diseased subjects, as well as in patients pre- and post-drug treatment.

One embodiment of this aspect of the invention therefore provides an array of antibodies, said array comprising at least two different antibody species, wherein each antibody species is immunospecific for a gene product of a gene implicated in endometriosis by the method of the first aspect of the invention. The term “immunospecific” as used herein means that the antibodies have substantially greater affinity for the polypeptides of the invention than their affinity for other related polypeptides in the prior art. As used herein, the term “antibody” refers to both polyclonal antibodies and monoclonal antibodies, to intact molecules as well as fragments thereof, such as Fab, F(ab′)₂ and Fc, which are capable of binding to the antigenic determinant in question. Such antibodies thus bind to the polypeptides identified according to the first aspect of the invention.

A further embodiment of this aspect of the invention also provides an array of polypeptides, said array comprising at least two polypeptide species, wherein each polypeptide species comprises a gene product of a gene implicated in endometriosis by the method of the first aspect of the invention, or is a functional equivalent or a fragment thereof. Functionally-equivalent polypeptides of the first aspect of the invention may be polypeptides that are homologous to the polypeptides explicitly identified herein. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar hydrophobicity and charge type between the sequences. Degrees of identity and similarity can be readily calculated by those of skill in the art. Natural biological variants and mutants are therefore homologues as this term is used herein.

Typically, greater than 50% identity between two polypeptides is considered to be an indication of functional equivalence. Preferably, functionally equivalent polypeptides of the first aspect of the invention have a degree of sequence identity with the polypeptide explicitly identified, or with active fragments thereof, of greater than 50%. More preferred polypeptides have degrees of identity of greater than 60%, 70%, 80%, 90%, 95%, 98% or 99%, respectively.

Fragments of the polypeptides, or of functional equivalents thereof may also be used on arrays according to this aspect of the invention. As used herein, the term “fragment” refers to a polypeptide having an amino acid sequence that is the same as part, but not all, of the amino acid sequence of the polypeptides explicitly identified, or with one of its functional equivalents. The fragments should comprise at least n consecutive amino acids from the sequence and, depending on the particular sequence, n preferably is 7 or more (for example, 8, 10, 12, 14, 16, 18, 20 or more). For instance, small fragments may form an antigenic determinant. Such fragments may be “free-standing”, i.e. not part of or fused to other amino acids or polypeptides, or they may be comprised within a larger polypeptide of which they form a part or region.

Nucleic acid, antibody and protein arrays as described above may be designed such that single arrays only contain nucleic acid and polypeptide molecules that belong to certain functional categories. Examples of such categories include proteases and protease inhibitors, tumour suppressor proteins, proteins of the immune system, proteins that are involved in an inflammatory response, enzymes, lipid binding proteins and or matrix or cell adhesion molecules.

Additional nucleic acids or proteins may be included on the array that are members of the same signalling pathway or metabolic pathway as the genes and proteins that have been explicitly identified herein as having a role in endometriosis. On an individual patient basis, using arrays such as these may clarify the exact point(s) in the pathway that is responsible for disease in the patient concerned. Alternatively, and particularly as the technology in this area develops, such that more nucleic acid molecules or polypeptides may be included on a single array, comprehensive arrays may be designed that include a large number of genes or protein types.

It is envisaged that the use of such arrays may facilitate the identification of other genes and proteins that are implicated in this disease. These genes and proteins, implicated in endometriosis, form a further aspect of the invention.

Detection of Mutations in Genes

The invention also encompasses methods for the detection of the presence or absence of a mutation or polymorphism associated with endometriosis in a nucleic acid product isolated from patient tissue. Examples of available methods used for the detection of mutations in DNA sequences include direct sequencing methods (Maxim & Gilbert, 1977 PNAS USA 74: 560-564; Sanger et al (1977) PNAS USA 74: 5463-5467), PCR methods, single-stranded conformational polymorphism (SSCP) based methods of analysis and gene arrays. Protection assays, such as the RNAse protection assay described by Meyers et al (1985, Science, 230(3): 1242-1246) are also suitable.

In one aspect of the invention, the presence or absence of a mutation in a subject in need of diagnosis of endometriosis is detected by contacting nucleic acid from a patient, encoding a gene implicated in endometriosis, with a probe that hybridises to the nucleic acid under stringent conditions to form a hybrid double-stranded molecule. The hybrid double-stranded molecule will have an unhybridised portion of the nucleic acid probe strand at any portion corresponding to a mutation associated with endometriosis. Accordingly, the detection of the presence or absence of an unhybridised portion of the probe strand gives an indication of the presence or absence of an endometriosis-associated mutation in the corresponding portion of the DNA strand.

For example, the presence or absence of a mutation in a patient in need of diagnosis of endometriosis may be detected by hybridisation of nucleic acid isolated from the patient on an oligonucleotide array (often termed a microarray). This will allow the rapid screening of single nucleotide polymorphisms in endometriosis-related genes, such as those genes implicated in endometriosis by the method of the first aspect of the invention. Oligonucleotide arrays will typically contain every oligonucleotide of specified length that represents the consensus gene sequence, as well as for the three permutations of each consensus oligonucleotide other than the wild type sequence, which incorporate single base changes at a specific nucleotide position. In this way, every possible one-base substitution variant of the gene may be represented on the chip (8N for a gene target of N length).

If more than one gene is to be probed for single nucleotide polymorphisms, then sets of oligonucleotides for each gene need to be arrayed. Examples of genes implicated herein in endometriosis include cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1 and podocalyxin.

According to this embodiment, a nucleic acid array may be used, identical or similar to those described in detail above. Such arrays may contain a plurality of nucleic acid molecules of overlapping sequence, each nucleic acid molecule consisting of a portion of the consensus sequence of a gene implicated in endometriosis, or its complement, wherein a nucleic acid molecule is included on the array that corresponds to each of the four potential nucleotide variants at a number of nucleotide positions in the gene sequence, the remainder of the nucleic acid molecule corresponding to the wild type sequence of the gene. A nucleic acid molecule may be included on an array that corresponds to each of the four potential nucleotide variants at every nucleotide position in the gene sequence.

Oligonucleotide arrays such as those described above will allow screening for single nucleotide polymorphisms (SNPs) in large numbers of patients, in order to determine genetic risk factors. SNPs so identified form a further aspect of the present invention.

Specific diagnostic methods according to the invention may include the steps of: a) contacting a sample of nucleic acid from a tissue of a patient with a nucleic acid probe or nucleic acid array under stringent conditions that allow the formation of a hybrid complex between the patient nucleic acid and the probe or array; b) contacting a control nucleic acid sample with said probe or array under the same conditions used in step a); and c) detecting the presence of hybrid complexes in said samples; wherein detection of levels of the hybrid complex in the patient sample that differ from levels of the hybrid complex in the control sample is indicative of endometriosis.

A similar method may comprise the steps of: a) contacting a sample of nucleic acid from tissue of a patient with a nucleic acid primer under stringent conditions that allow the formation of a hybrid complex between the patient nucleic acid and the probe; b) contacting a control sample with said primer under the same conditions used in step a); c) amplifying the sampled nucleic acid using the primers; and d) detecting the level of the amplified nucleic acid from both patient and control samples; wherein detection of levels of the amplified nucleic acid in the patient sample that differ significantly from levels of the amplified nucleic acid in the control sample is indicative of endometriosis.

A further method may comprise the steps of: a) obtaining a tissue sample from a patient being tested for endometriosis; b) isolating nucleic acid encoding a gene identified according to the method of the first aspect of the invention from said tissue sample; and c) assessing the risk of a patient developing endometriosis on the basis of the presence or absence of a mutation in the nucleic acid sample which is associated with endometriosis. A further step in this method may comprise the step of amplifying the DNA to form an amplified product and detecting the presence or absence of a mutation in the amplified product which is associated with endometriosis.

The presence or absence of the mutation in the patient may be detected by contacting patient nucleic acid with a nucleic acid probe that hybridises to said patient nucleic acid under stringent conditions to form a hybrid double-stranded molecule, the hybrid double-stranded molecule having an unhybridised portion of the nucleic acid probe strand at any portion corresponding to a mutation associated with endometriosis; and detecting the presence or absence of an unhybridised portion of the probe strand as an indication of the presence or absence of an endometriosis-associated mutation in the corresponding portion of the DNA strand. The unhybridised portion of the probe strand may be detected by contacting the hybrid double-stranded molecule with an agent capable of digesting an unhybridised portion of the hybrid double-stranded molecule.

Diagnosis of Aberrant Protein Expression

Alternatively, the gene product levels may be determined. For example, if the gene product of interest is an enzyme, an enzyme assay may be performed. If an antibody to the gene product is available, methods for specific protein determination may use these antibodies in technologies such as immunohistochemistry, immunocytochemistry or ELISA-based methods. Enzymatic or ELISA assays are suitable for determining gene products of relatively abundant or stable mRNA moieties. Other assays based on binding properties include binding assays using biosensors, for example (BIACORE™).

According to a further aspect of the invention there is provided a method for detecting or diagnosing endometriosis in a patient, comprising assessing the level of a gene product which is associated with endometriosis in tissue from said patient (such as ectopic endometrial tissue) and comparing said level of expression to a control level of expression wherein a level of expression that differs from said control level is indicative of susceptibility of endometriosis disease. Preferably, a control level of expression is the level found in healthy endometrium tissue from the patient. Suitable tissues for biopsy include those tissues listed above as well as blood.

Abnormal levels of gene expression may be monitored by altering the levels of gene product or by assessing biological activities of gene product, for example, by assessing levels of phosphorylation, synthesis or metabolic degradation. An abnormality in the activity or level of the gene product is an indication of disease. The term “abnormality” may include an inability of the protein to perform its intended biological function in the body. This term is also intended to include an abnormal stoichiometry in the level of protein, the expression of a non-functional or partially functional form of the wild type protein and total lack of expression of the protein.

Any agent that is capable of binding specifically to the gene product will be useful in this aspect of the invention. Typically, antibodies will be used. Methods of generation of monoclonal and polyclonal antisera are discussed above. Typically, tissue for testing is obtained from a patient by biopsy and is then contacted with antibody, using conventional techniques of immunocytochemistry or immunohistochemistry. ELISA techniques, more preferably competitive ELISA techniques, are particularly suitable and could be used for testing either tissue homogenates or blood samples from patients.

In some instances, it may be preferable to perform diagnosis in situ in the patient. This would avoid the need for invasive measures of obtaining tissue for biopsy such as laparoscopy. For such an application, hybrid molecules may be used, comprising a composite of one or more polypeptides and a second agent such as a radiolabel or chemical compound or a green fluorescent protein variant fusion. When applied to a patient, the molecule targets the site of disease through the specificity of a binding portion of the molecule for the gene product implicated in endometriosis. The label portion allows visualisation of diseased areas, for example by detection of gamma or beta particles or photons of emitted light, thus allowing the physician to assess the extent and severity of disease. Suitable methods of labelling will be known to those of skill in the art and include the use of radiolabels and fluorescent labels.

In vivo visualization of gene expression in diseased tissues using magnetic resonance imaging (MRI) provides an alternative way to visualise diseased areas (Nature Biotechnology, 2000, Vol.18, Louie A Y et al.). When applied to a patient, the MRI contrast agent containing substrate for a target molecule gets cleaved by the target molecule at the disease site, so allowing a paramagnetic ion to interact directly with water protons and to increase the magnetic resonance signal.

According to a further aspect of the invention, a gene selected by the method of the first aspect of the invention, or the protein product of said gene may be used in a screening method to identify other genes or proteins that are implicated in endometriosis, for example by screening a gene or a protein library. This aspect of the invention therefore provides a method for the identification of an agent that is effective in the treatment and/or diagnosis of endometriosis, comprising contacting a gene or gene product as described above with one or more compounds suspected of possessing affinity for said gene or gene product, and selecting as said agent, a compound that binds to said gene or gene product.

Such a screening method may also be used to identify candidate drugs or other compounds that are effective in treating or diagnosing endometriosis. For example, screening could be performed using drugs or combinatorial libraries of compounds with potential to modulate protein or gene activity in order to identify agents that are effective in binding to genes or gene products initially identified as having a role in endometriosis in the differential expression screen discussed above. Such agents may be used as the basis for identifying compounds with therapeutic and/or diagnostic potential.

In particular, the use of gene arrays is considered particularly promising, both for the diagnosis of endometriosis and for the identification of additional genes and gene products that are implicated in endometriosis. In one respect, arrays might contain nucleotide sequences corresponding to the sequences of genes initially implicated in endometriosis by the differential screening process discussed above. Such arrays would considerably facilitate the screening of large numbers of individuals at low cost. Detailed information regarding the design and construction of arrays may be found in the art, particularly in the following documents: U.S. Pat. No. 5,925,525; U.S. Pat. No. 5,922,591, WO99/35256; WO99/09218; WO98/56954; U.S. Pat. No. 5,837,832 and U.S. Pat. No. 5,770,722.

Furthermore, it is considered that the use of arrays that contain gene sequences encoding proteins in the same metabolic or signalling pathway as the gene initially implicated in endometriosis by the differential screening process will allow the identification of additional targets for the diagnosis and therapy of this disease. Arrays of this kind allow the quantification of the levels of a particular gene transcript, meaning that quite precise evaluations of gene expression become possible.

### THERAPY

In order to treat endometriosis in a patient, a number of approaches are possible. As the skilled person will appreciate, the choice of approach will depend upon the nature of the gene whose aberrant expression has been implicated in disease. According to the invention, methods for treating endometriosis generally involve adjusting in the patient an abnormal stoichiometry of a protein implicated in endometriosis, so as to revert to the normal stoichiometry that is reflected in the patient's healthy tissue. The term “patient” for the purposes of this invention is intended to include any mammalian subject capable of being afflicted with endometriosis. Preferred patients are humans.

Examples of genes and proteins implicated in endometriosis by the method of the first aspect of the invention include cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1 and podocalyxin.

In one aspect of this embodiment of the invention, there is provided a method of treating endometriosis in a patient comprising administering to the patient a compound that is effective to alter the expression of a gene or gene product selected by the method of the first aspect of the invention, in a relevant tissue in the patient, such as endometrium tissue. The invention also provides compositions that are useful for the amelioration or correction of endometriosis symptoms, comprising a gene or gene product selected by the method of the first aspect of the invention, or an antagonist or agonist of said gene or gene product. By agonist is meant herein, any polypeptide, peptide, synthetic molecule or organic molecule that functions as an activator, by increasing the effective biological activity of the gene product, for example, by increasing gene expression or enzymatic activity. By antagonist is meant herein, any polypeptide, peptide, synthetic molecule or organic molecule that functions as an inhibitor, by decreasing the effective biological activity of the gene product, for example, by inhibiting gene expression of an enzyme or a pharmacological receptor.

In some cases, the most appropriate target for treatment will be the identified gene itself, using agents that are specifically effective in either increasing or decreasing levels of expression of the gene implicated in endometriosis by the method of the first aspect of the invention.

Of course, a combination of genes, gene products, agonists and/or antagonists may be used, if appropriate. The necessity to use a combination of approaches for a particular therapy, or in connection with a particular patient will be clear to the clinician and quite within the abilities of the skilled reader. In some circumstances, it may be that a degree of trial and error will be needed in order to design an effective therapeutic regime. However, once the genes involved in endometriosis have been identified, it is thought that this degree of analysis and testing is unlikely to involve the application of inventive thought.

Inhibition of Gene Expression

A variety of techniques may be utilised to inhibit the expression, synthesis, or activity of genes implicated in endometriosis.

For example, one mechanism is by using antisense nucleic acid molecules that inhibit expression of the gene by binding to the genomic DNA or to the mRNA transcribed from the gene. The production and use of antisense molecules is well known to those of skill in the art (see, for example, Robinson et al., (1996) P.N.A.S. USA 93: 4851). Antisense molecules may act by directly blocking the translation of transcribed mRNA by binding specifically to mRNA species and preventing protein translation. Another target for binding to an mRNA molecule is in the environment of the ribosome initiation site, although any target site may be used that is effective in preventing mRNA translation. Antisense oligonucleotides may also be targeted to areas of the genome itself, to inhibit or prevent gene transcription. Promoter sequences are particularly advantageous targets in this respect.

Specificity is attained by using oligonucleotides of defined sequence that are complementary to a portion of the gene sequence. It is preferable to use RNA or DNA oligonucleotides of between 15 and 30 nucleotides length to ensure that the targeting is specific and effective.

Antisense molecules may be stabilised by way of modifications such as by the addition of flanking sequences of ribodeoxynucleotides to the 5′ and/or 3′ ends of the molecule, or the use of phosphorothioate or 2′ O-methyl rather than phosphodiester linkages within the oligodeoxyribonucleotide backbone.

Ribozyme molecules are also effective agents for the specific abolition of gene expression, most suitably targeting the mRNA transcribed from the gene whose aberrant expression is implicated in endometriosis. The design of ribozymes targeted against specific gene molecules is known in the art (see, for example, Perriman and Gerlach (1990) Curr Opin Biotechnol. 1(1): 86-91; Welch et al., (1998) Curr Opin Biotechnol. 9(5) 486-496; Gibson and Sillitoe (1997) 7(2): 125-137). Specificity is attained using “arms” that are complementary to the sequence of the gene target. The mechanism of action of ribozymes involves sequence-specific hybridisation of part of the ribozyme molecule to a complementary target RNA, after which event the molecule is cleaved.

Triplex DNA formation is another method that has been shown to be effective in the specific inhibition of gene expression. The theory behind this concept is that nucleotides can bind specifically via non Watson-Crick interactions to this gene and interrupt the action of transcriptase enzymes as they progress along the genomic DNA. Further details may be found in Chan and Glazer (1997, J. Mol. Biol. 75(4): 267-228).

Protein may also be used to alter the level of expression of a gene whose aberrant expression is associated with endometriosis. For example, DNA binding proteins that bind specifically to promoter areas of the gene may decrease or increase the expression of a gene. Examples include the various types of hormone response elements that are effective to increase expression of a gene upon binding of a hormone receptor complex to the appropriate element on the genome.

Enhancement of Gene Expression

Similar techniques exist that are effective to increase the expression of a gene whose aberrant expression is implicated in endometriosis, as the skilled artisan will appreciate.

For example, gene replacement therapy allows substitution of the aberrant gene with the healthy wild type gene. The entire gene may be replaced or, alternatively, only the portion of sequence which is causative of endometriosis disease. Replacement genes may also comprise the non-coding areas of the gene, for example, in instances in which it is the regulation of gene expression that is defective. As the skilled artisan will appreciate, numerous techniques now exist to effect substitution of a copy of the normal gene or a portion of the gene for the aberrant gene.

To be effective, replacement gene therapy must inhibit expression of the mutant gene and provide normal function of the same gene simultaneously in endometrial tissue, since it is in these cells that defective expression is causative of endometriosis disease. Suitable techniques for the introduction of gene therapy vehicles into cells include electroporation, the use of DNA guns, direct injection of pure nucleic acid into endometrial tissue and liposome-mediated techniques (see, for example, Dachs et al., (1997) Oncol Res. 9(6-7): 313-325; Templeton and Lasic (1999) Mol Biotechnol 11(2): 175-180; Ozaki et al., (1999) J. Hepatology 31(1), 53-60). Such vehicles can be administered either locally or systemically.

Therapeutic DNA sequences may be administered by any suitable technique such as, for example, using liposome formulations or recombinant viral vectors. Suitable viral vectors include, for example, vectors derived from retroviruses, adenoviruses, adeno-associated viruses, herpes viruses or papilloma viruses. Non-viral vectors include simple plasmids formulated, for example, as liposomes (Templeton and Lasic, 1999; Maurice et al., (1999) J. Clin. Invest. 104(1): 21-29). Expression of the coding sequence can be induced using endogenous mammalian or heterologous promoters, and may be either constitutive or regulated.

A further form of replacement gene therapy involves the introduction of cells, preferably endometrial cells, most preferably autologous endometrial cells that contain wild type gene sequences. Reversal of disease by implantation of cells genetically-engineered to release mature protein has already been achieved in mice and could potentially be developed for humans (Falqui L et al. Hum. Gene Ther. 10 (11) 1753-1762). The cells may comprise autologous cells harvested from the patient and transfected ex vivo with one or more replacement genes (Bailey C. J. et al., (1999) J. Mol. Med. 77(1) 244-249). Transplantation of these cells back into the patient in areas that allow for the amelioration of endometriosis symptoms may restore the healthy function of the endometrial tissue and so prevent disease progression.

Gene therapy may also involve the introduction into a patient of a gene that is not itself implicated in endometriosis, but which effects an increase or decrease in the expression of a gene where aberrant expression is causative of this disease. The mechanism may be by causing a change in the degree of transcription of the gene, by altering the mRNA stability of the gene, or by any other indirect mechanism that restores the normal stoichiometry of the expressed gene that is replaced in healthy endometrium tissue.

Targeting Protein Expression

Rather than targeting gene expression, therapy to ameliorate or prevent the symptoms of endometriosis may be targeted to the gene product expressed from the gene whose aberrant expression is implicated in endometriosis. Such methods of therapy should be aimed at restoring the normal stoichiometry of the gene product in the diseased tissue relative to the levels in healthy tissue.

For gene products whose levels are higher than normal in the diseased tissue, an antagonist of the gene product may be used to treat the patient. Such antagonists may be directed against the biological activity of the gene product, for example, by inhibiting an enzymatic activity of the gene product. In this manner, the aberrant activity may be restored to the levels of healthy tissue.

Another mechanism by which abnormal biological activity may be corrected is by using an antagonist agent that binds to the gene product and prevents it from performing its normal physiological function. By binding in this fashion, the antagonist agent acts to titrate out the biological activity of the gene product, so restoring the normal stoichiometry that is present in healthy tissue.

Antibodies may be used as antagonist agents directed against gene products implicated in endometriosis. In this aspect of the invention, the antibodies may act by preventing binding of a ligand of the gene product, so destroying the biological activity of the gene product on its ligand. Alternatively, the antibody may target the gene product for destruction by the immune system, so removing it from circulation. These mechanisms, and modifications thereof, will be clear to those of skill in the art.

Antibodies may be generated that are specific for certain polymorphic gene product variants. For example, in some instances, it may be desirable to target only aberrant gene products rather than the wild type, unmutated gene products that perform valuable physiological functions in areas of the body other than the diseased tissue.

Antibodies may be prepared by conventional methods, for example by immunisation of animals such as mice, rats, rabbits, horses, sheep or goats. Rabbits, horses, sheep and goats are preferred for the preparation of polyclonal sera due to the volume of serum obtainable and the availability of labelled anti-rabbit, anti-horse, anti-sheep and anti-goat antibodies. Immunisation is generally performed by mixing or emulsifying the protein in saline, preferably in an adjuvant such as Freund's complete adjuvant, and injecting the mixture or emulsion parenterally, for example by intravenous, intraperitoneal, subcutaneous or intramuscular administration. rmmunisation might also be accomplished by immobilising a peptide epitope on a carrier protein and injecting the preparation into the animal.

Monoclonal antibodies against a gene product may be prepared using the standard method of Kohler & Milstein (Nature (1975) 256:495-96), or by a modification thereof. Typically, the spleen of an immunised mouse or rat is removed and dissociated into single cells. The cells are then induced to fuse with myeloma cells to form hybridomas, and are cultured in a selective medium such as hypoxanthine, aminopterin, thymidine (HAT) medium. The resulting hybridomas are plated by limiting dilution and are assayed for the production of antibodies that bind specifically to the immunising gene product. The selected hybridomas may then be cultured either in vitro or in vivo, for example, as ascites in mice.

Recombinant methods for producing monoclonal Fab fragments of a desired specificity are available in the art and have been successfully used to obtain high-affinity functional and clinically-applicable human monoclonal antibodies capable of differentiating diseased areas from healthy areas (Huls G. A. et al. Nat Biotechnology (1999) 17(3): 276-281; Burioni R et al., J Immunol Methods (1998) 217(1-2): 195-199).

Chimeric antibodies, in which different portions of the molecule are derived from different animal species or from different proteins, may also be used in this aspect of the invention, as can antibodies that contain grafted domains that are effective to reduce the amount of antibody recognised as foreign by host immune system. Antibody fragments that recognise specific epitopes on gene products implicated in endometriosis may also be used. Such fragments include, for example, Fab fragments or single chain antibody fragments produced according to standard methods.

For example, a chimeric antibody may contain a specific binding entity that directs the molecule to a target of endometriosis disease, such as by binding to a gene product that is implicated in endometriosis. A second entity in the hybrid molecule may show specificity for a surface marker on an immune cell and therefore act as a target for attack by elements of the immune system such as leukocytes, lymphocytes or mast cells.

In order for this method to work, chimeric molecules must be targeted to diseased cells. This can be effectively achieved by targeted gene therapy, as will be clear to those of skill in art. Effector cells may thus be attracted to the site of disease. Expression of the hybrid molecule in a diseased cell will then ensure its destruction. A particularly suitable combination of ligand specificities is anti-CD3 with anti-CD28, to recruit and stimulate T-cells.

Enhancement of Protein Expression

In one method for the restoration of gene product levels, the gene product itself may be administered to a patient suffering from the disease in an amount sufficient to ameliorate or prevent endometriosis symptoms. This method of therapy involves treating the patient with replacement amounts of the gene product whose levels or whose biological activity is depressed in diseased tissue. The replacement gene product may be applied systemically, although in most instances, the most suitable mode of administration will be to the area affected by disease, such as the ovaries, vagina, peritoneum, recto-vagina or any other ectopic site that contains lesions characteristic of endometriosis disease.

The gene product may be purified from natural sources, may be chemically synthesised or may be recombinantly expressed. Methods for the purification of gene product from natural sources will depend largely on the nature of the gene product and will in most instances either be known in the art or will be within the abilities of the skilled person to establish. Methods for the chemical synthesis of polypeptides or their fragments are well known to those of skill in the art (see, for example, Creighton, 1983, Proteins: Structures and Molecular Principles).

Preferably, the gene product will be prepared recombinantly, allowing high levels of expression at an economic cost. Recombinant expression is widely known in the art and involves the incorporation of the gene encoding the gene product of interest into an expression vector. Such an expression vector will incorporate appropriate transcriptional and translational control sequences, for example enhancer elements, promoter-operator regions, termination stop sequences, mRNA stability sequences, start and stop codons or ribosomal binding sites, linked in frame with the gene encoding the gene product. Secretion signalling and processing sequences may also be appropriate. Many suitable vectors and expression systems are well known and documented in the art (see, for example, Fernandez & Hoeffler, eds. (1998) Gene Expression Systems. Using Nature for the Art of Expression). Particularly suitable viral vectors include baculovirus-, adenovirus- and vaccinia virus-based vectors.

Gene products may be expressed recombinantly in prokaryote hosts, such as in E. coli, or in eukaryotic yeasts that can be made to express high levels of recombinant proteins and that can easily be grown in large quantities. Mammalian cell lines grown in vitro are also suitable, particularly when using virus-driven expression systems. Another suitable expression system is the baculovirus expression system that involves the use of insect cells as hosts. An expression system may also constitute host cells that have the encoding DNA incorporated into their genome. Recombinant protein may easily be purified from these hosts in large quantities and at an economic cost.

An alternative method for the correction of the levels of gene products that are lower in diseased tissue than the level found in healthy tissue is by applying to the patient, preferably in a diseased area, an agonist of the gene product. By agonist is meant any polypeptide, peptide, synthetic molecule or organic molecule that functions as an activator in the diseased tissue, to increase the effective biological activity of the gene product and to restore normal physiological biological activity of the gene product in the diseased tissue.

One method of increasing biological activity of the gene product is to increase the effective concentration of gene product. This may be done in several ways, for example, by using an antibody as the agonist agent, that acts to titrate out the level of a natural antagonist or regulator of the gene product. This leaves more free gene product available to function in its natural role. In this fashion, the symptoms of endometriosis may be ameliorated or prevented altogether.

Pharmaceutical Preparations

Gene products and agonist and antagonist compounds for the treatment of endometriosis will generally be administered to patients as pharmaceutical compositions in therapeutically-effective doses. The term “therapeutically-effective dose” as used herein refers to an amount of a therapeutic agent that is effective to treat, ameliorate, or prevent endometriosis, or to exhibit a detectable therapeutic or preventative effect. The precise effective amount for a subject will depend upon the subject's size and health, the route of administration, the nature and extent of the disease condition, and the therapeutic agent or combination of therapeutic agents selected for administration.

The effective dose for a given situation can be determined by routine experimentation and is within the judgement of the skilled person. For example, in order to formulate a range of dosage values, cell culture assays and animal studies can be used. The dosage of such compounds preferably lies within the dose that is therapeutically effective in 50% of the population, and that exhibits little or no toxicity at this level. For purposes of the present invention, an effective dose will be between 0.01 mg/ kg and 50 mg/kg or, more typically, between 0.05 mg/kg and 10 mg/kg of the individual to which it is administered.

Pharmaceutical compositions may also contain a pharmaceutically acceptable carrier. Suitable carriers may be large, slowly metabolised macromolecules such as proteins, polysaccharides, polylactic acids, polyglycolic acids, polymeric amino acids, amino acid copolymers and inactive virus particles. Pharmaceutically acceptable salts may also be used, for example, mineral acid salts such as hydrochlorides, hydrobromides, phosphates, and sulphates, or salts of organic acids such as acetates, propionates, malonates, benzoates (see Remington's Pharmaceutical Sciences (Mack Pub. Co., N.J. 1991)).

Pharmaceutically acceptable carriers in therapeutic compositions may also contain liquids such as water, saline, glycerol and ethanol. Additionally, auxiliary substances, such as wetting or emulsifying agents and pH buffering substances, may be present.

In addition to the inclusion of a carrier, the pharmaceutical compositions of the invention may optionally include a delivery vehicle. Typically, the pharmaceutical compositions will be prepared as injectables, either as liquid solutions or suspensions. Recently, patches have been developed as a method of effecting the continuous administration of drugs. Solid forms suitable for solution in, pessaries, or suspension in liquid vehicles prior to injection may also be prepared. Preparations for oral administration may also be formulated to allow for controlled release of the active compound, for example, using capsules or cartridges. For administration by inhalation, the compounds of the invention may be delivered by any means known to those of the skill in the art, including, for example, using aerosol sprays. Considering the nature of the disease, vaginal delivery systems, such as medicated tampons, gels, sprays, and other devices may also be used for administering compounds near the endometriosis affected regions.

Kits

The invention also relates to kits that are useful for diagnosing endometriosis. Such kits may be suitable for detection of nucleic acid species, or alternatively may be for detection of a gene product, as discussed above.

For detection of nucleic acid, such kits may contain a first container such as a vial or plastic tube or a microtiter plate that contains an oligonucleotide probe. The kits may optionally contain a second container that holds primers. The probe is hybridisable to DNA whose aberrant expression is associated with endometriosis and the primers are useful for amplifying this DNA. Kits that contain an oligonucleotide probe immobilised on a solid support could also be developed, for example, using arrays (see supplement of issue 21(1) Nature Genetics, 1999, the references cited therein and the references cited above).

In another embodiment, the kits may contain an agent that digests single-stranded nucleic acid or RNA and/or instructions for using the components of the kits to detect the presence or absence of an endometriosis-associated mutation in amplified DNA.

For PCR amplification of nucleic acid, nucleic acid primers may be included in the kit that are complementary to at least a portion of a gene that encodes a protein associated with endometriosis. The set of primers typically includes at least two oligonucleotides, preferably four oligonucleotides, that are capable of specific amplification of DNA. Fluorescent-labelled oligonucleotides that will allow quantitative PCR determination may be included (e.g. TaqMan chemistry, Molecular Beacons). Suitable enzymes for amplification of the DNA, will also be included.

Control nucleic acid may be included for purposes of comparison or validation. Such controls could either be RNA/DNA isolated from healthy tissue, or from healthy individuals, or housekeeping genes such as β-actin or GAPDH whose mRNA levels are not affected by endometriosis disease.

For detection of gene product, antibodies will most typically be used as components of kits. However, any agent capable of binding specifically to the gene product will be useful in this aspect of the invention. Other components of the kits will typically include labels, secondary antibodies, substrates (if the gene is an enzyme), inhibitors, co-factors and control gene product preparations to allow the user to quantitate expression levels and/or to assess whether the diagnosis experiment has worked correctly. Enzyme-linked immunosorbent assay-based (ELISA) tests and competitive ELISA tests are particularly suitable assays that can be carried out easily by the skilled person using kit components.

Transgenic Animals

The invention also provides a genetically-modified non-human animal that has been transformed to express higher, lower or absent levels of a gene or gene product identified by the method of the first aspect of the invention. Preferably, genetically-modified animal is a transgenic animal, designed to express aberrant levels of the gene, most preferably in endometrium tissue. Alternatively the animal may include a deletion of, or a deletion in the gene of interest (knockouts). Such animals are useful as screens for pharmacologic agents effective in the treatment of endometriosis.

Methods for the production of genetically-modified animals are known in the art and include techniques such as modification of somatic cells, or germ line therapy to incorporate heritable modifications (see, for example, Rajewsky et al., (1996), J Clin Invest 98, 600-3; Metzger and Feil, (1999) Curr. Opinions Biotechnology 10, 470-476; Bedell et al. (1997), Genes Dev. 11: 11-43; Bedell et al (1997), Genes Dev. 11: 1-10; “Transgenic Mammals”, John Bishop (1999) Pearson Education Ltd., Harlow, Essex, for example, p228). Preferably, transgenic organisms are created using germ line gene therapy.

According to a still further aspect of the invention, there is provided a method for screening for an agent for an ability to treat endometriosis, by contacting a genetically-modified non-human animal as described above, comprising DNA whose aberrant expression has been associated with endometriosis, with a candidate agent and determining the effect of the agent on the endometriosis disease of the animal.

Proteins Implicated in Endometriosis

According to certain specific aspects of the invention, it has been discovered that proteins with a wide variety of different functions have a role in endometriosis. These functions may be classified into broad classes, such as proteases and protease inhibitors, tumour suppressors, proteins involved in cell growth and proliferation, matrix and cell adhesion related molecules, genes and proteins with a function in the immune system or in inflammatory processes, lipid binding proteins, enzymes and transcription/translation regulatory factors.

The invention therefore provides a protease, or active fragment thereof; a tumour suppressor protein or active fragment thereof; a protein involved in cell growth and proliferation, or an active fragment thereof; a matrix and cell adhesion protein, or active fragment thereof; a protein with a function in the immune system or in inflammation, or active fragment thereof, a lipid binding protein or active fragment thereof, an enzyme or active fragment thereof, a globulin or active fragment thereof or a transcription/translation regulatory factor or active fragment thereof; or a gene encoding a protease or protease fragment; a tumour suppressor protein or fragment of a tumour suppressor protein; a protein involved in cell growth and proliferation or fragment of a protein involved in cell growth and proliferation; a gene encoding a matrix or cell adhesion protein or fragment of a matrix protein or cell adhesion protein; or a gene encoding a protein with a function in the immune system or inflammation or a fragment of a protein with a function in the immune system or inflammation, a lipid binding protein or active fragment thereof, an enzyme or active fragment thereof, a globulin or active fragment thereof or a transcription/translation regulatory factor or active fragment thereof, for use in the manufacture of a medicament for the treatment or diagnosis of endometriosis.

Such molecules, or agonists and antagonists of these molecules, may also be used in methods of treatment of patients suspected of suffering from endometriosis.

By “active fragment” is meant herein any fragment of a gene product that retains a biological activity that is associated with the full length gene product.

Specific examples of proteases and protease inhibitors herein implicated in endometriosis include cathepsin D, AEBP- 1, stromelysin-3, cystatin B and protease inhibitor 1. Cathepsin D and AEBP-1 are expressed at higher than normal levels, whilst cystatin B, protease inhibitor 1 and stromelysin-3 are primarily present at lower levels or are completely absent in diseased tissue.

Specific examples of tumour suppressor genes implicated in endometriosis include those that make up the Wnt signalling pathway. This pathway consists of frizzled receptors that belong to the family of seven transmembrane receptors, the ligands of these receptors and several intracellular downstream proteins that transduce the signal from the cell surface to the nucleus. Wnt proteins interact with frizzled receptors and the signal is transduced through the dishevelled (Dvl-1) proteins. Dvl proteins inhibit glycogen synthase kinase 3 beta, leading to stabilisation and accumulation of cytosolic b-catenin. b-catenin then binds to the lymphoid enhancing factor 1 (Lef-1)/T cell factor (Tcf) family of transcription factors and the complex translocates to the nucleus where gene transcription is activated. A family of secreted frizzled related proteins (sFRP or Fritz, Frzb and SARP) antagonise the Wnt pathway by binding to Wnt and inhibiting it from binding to its receptor.

The discovery reported herein, of down-regulation of the secreted frizzled protein in endometriosis, implicates the entire Wnt pathway in endometriosis. Therefore, proteins such as wnt, frizzled, Dvl-1, and axin, and inhibitors or activators of the enzymes directly involved in the pathway such as glycogen synthase kinase 3 beta, casein kinase I (Peters J M et al. Nature, (1999) 401, 345-350) as well as transcription factor (Lef/Tcf family) modulators provide points of intervention for treatment or diagnosis of endometriosis.

In particular, the gene encoding the Wnt antagonist sFRP4 (secreted frizzled related protein 4), also known as frizzled related protein frpHE, is implicated herein in the disease endometriosis. The level of this protein is lower in diseased tissue from patients with endometriosis.

Gelsolin, a plasma protein and a tumour suppressor gene unrelated by sequence to sFRP4 is also implicated herein in this disease. The level of this protein is higher in diseased tissue from patients with endometriosis.

Specific examples of proteins involved in cell growth and proliferation herein implicated in endometriosis include the insulin-like growth factor-binding protein-3 (IGFBP-3), that is shown herein to be downregulated in endometriotic tissue and dual specificity phosphatase 1 also known as MKP-1 which is upregulated in endometriotic tissue.

Specific examples of immune-system-related proteins herein implicated in endometriosis include the immunoglobulin λ chain, human progestagen-dependent pregnancy-associated endometrial protein (PAEP), complement component 3 and ferritin. PAEP has been found to be expressed at high levels in eutopic endometrium and to be completely absent in endometriosis in diseased individuals. Immunoglobulin λ, complement component 3 and ferritin are both found to be expressed at elevated levels in ectopic tissue from diseased individuals.

Specific examples of matrix and cell adhesion related proteins implicated in endometriosis include pro-alpha-1 type III collagen, alpha-2 type I collagen, procollagen C-endopeptidase enhancer, proline 4-hydroxylase, melanoma adhesion molecule and claudin-4. Melanoma adhesion molecule (MCAM; also known as CD146) is shown herein to be upregulated in endometriotic lesions. Alpha-2 type I collagen has been found to be upregulated in endometriotic lesions during the late secretory and early proliferative phase of the cycle. Collagen alpha-1 type 3, proline 4-hydroxylase beta polypeptide, claudin 4 and procollagen C-endopeptidase enhancer have been shown herein to be downregulated in endometriotic tissues.

Specific examples of other proteins implicated in endometriosis, falling into the categories of lipid binding proteins, enzymes and transcription/translation regulatory factors include elongation factor-1 alpha subunit, nascent polypeptide-associated complex alpha polypeptide, vitamin D3 25 hydroxylase, cysteine and glycine rich protein (CSRP-1), steroidogenic acute regulatory protein, apolipoprotein E, transcobalamin II, and prosaposin. Additionally, the transcription factor early growth response 1 (EGR1); ribosomal protein S6; the RNA editing enzyme adenosine deaminase; RNA-specific protein (ADAR); the DNA-double strand break repair gene RAD21; the PKC binding guanine nucleotide binding protein, beta polypeptide 2-like 1 (RACK1); and the endothelial cell surface protein podocalyxin have been found herein to play a role in endometriosis.

The sequences of the genes encoding these proteins have the accession numbers: sFRP4-gb: AF026692, gelsolin—X04412, IGFBP-3—M35878, dual specificity phosphatase 1 NM_{—}004417, cathepsin D—M11233; AEBP-1—D86479, stromelysin-3—emb x57766 and NM_{—}005940, cystatin B—AF208234, protease inhibitor 1—X01683, PAEP—emb: J04129, immunoglobulin λ—emb: Y14737, complement component 3—NM_{—}000064, ferritin L chain—M11147 and H chain—M11146, pro-alpha-1 type III collagen—NM_{—}000090 and emb: x14420, procollagen C-endopeptidase enhancer—NM_{—}002593, proline 4-hydroxylase beta polypeptide—NM_{—}000918, collagen alpha-2 type I—NM_{—}000089, claudin-4—NM_{—}001305, melanoma adhesion protein—NM_{—}006500, elongation factor I alpha subunit—emb:—x03558, nascent-polypeptide-associated complex alpha polypeptide—AF054187, vitamin D3 25 hydroxylase—emb: x59812, apoE—X00199, prosaposin—J03015, steroidogenic acute regulatory protein—NM_{—}000349, transcobalamin II—NM_{—}000355, CSRP-1—M33146, Early growth response 1 (EGR1)—NM_{—}001964, ribosomal protein S6—NM_{—}001010, adenosine deaminase RNA-specific protein—NM_{—}015841, RAD21—NM 006265, RACK1—NM_{—}006098, and podocalyxin—NM_{—}005397. The literature contains multiple names for some of these proteins. The accession numbers should be considered the unique description.

Nucleic acid molecules containing sequence derived from these genes, sequences that are complementary to these gene sequences, the products of these genes, and agonists and antagonists of these gene products may be used in any of the methods of therapy and diagnosis that are described herein.

Included in this aspect of the invention is the use of fragments of the cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1 and podocalyxin gene sequences, nucleic acid molecules that hybridise to these gene sequences and nucleic acid molecules that encode gene products which are functionally equivalent to the cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1 or podocalyxin gene products, in the manufacture of a medicament for the treatment or diagnosis of endometriosis.

By “functionally equivalent” is meant a gene product or peptide having the biological function of the cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1or podocalyxin gene products. Included as functional equivalents are gene products that exhibit significant sequence homology to the sequences of these proteins, such that they may be included within the same functional family. Two proteins are said to be “homologous”, as the term is used herein, if the sequence of one of the proteins has a high enough degree of identity or similarity to the sequence of the other protein. Homologous proteins therefore include natural biological variants (for example, allelic variants or geographical variations within the species from which the proteins are derived) and mutants (such as mutants containing amino acid substitutions, insertions or deletions) of the gene products explicitly identified herein. Typically, greater than 50% identity between two proteins is considered to be an indication of functional equivalence.

Preferably, functionally equivalent proteins of the first aspect of the invention have a degree of sequence identity with the protein explicitly identified herein, or with active fragments thereof, of greater than 50%. More preferred proteins have degrees of identity of greater than 60%, 70%, 80%, 90%, 95%, 98% or 99%, respectively.

Such functionally equivalent gene products may contain deletions, additions or substitutions of amino acid residues within the wild type gene product sequence, but which result in a conservative or silent nucleotide or amino acid substitution.

Pharmaceutical compositions comprising cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1and/or podocalyxin gene sequences, gene products, and agonists and antagonists of these gene sequences and/or gene products form a further aspect of the invention. Pharmaceutical compositions are discussed generally in some detail above.

Preferably, nucleic acids useful in this aspect of the invention should hybridise to a cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamnin II, prosaposin, early growth response 1 (EGR1), ribosomal protein S6, adenosine deaminase RNA-specific protein, RAD21, RACK1or podocalyxin gene sequence. Most suitably, conditions used will be those that avoid misdiagnosis and will generally be conditions of high stringency, for example 2×SSC, 65° C., 0.3% SDS (SSC=0. 15M NaCl, 0.015M sodium citrate, pH 7.2). In the case of gene arrays, conditions of high stringency may be 3×SSC, 0.3% SDS (see, for example, Cheung et al., (1999) Nat Genetics 21(1) suppl. pp 15-19).

The invention also includes methods of detecting mutations in the genes encoding cathepsin D, AEBP-1, stromelysin-3, cystatin B, protease inhibitor 1, sFRP4, gelsolin, IGFBP-3, dual specificity phosphatase 1, PAEP, immunoglobulin λ, complement component 3, ferritin, pro-alpha-1 type III collagen, proline 4-hydroxylase, alpha-2 type I collagen, procollagen C-endopeptidase enhancer, claudin-4, melanoma adhesion protein, elongation factor, nascent-polypeptide-associated complex alpha polypeptide, vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, RACK1and podocalyxin in tissue (including blood) from a patient suffering from endometriosis, to allow diagnosis of this disease, or of susceptibility to this disease.

As used herein, the term “mutation” is intended to include mutations associated with endometriosis. The mutation may be a gross alteration in the RNA or DNA of a patient or a small alternation, such as a point mutation. Examples of common mutations are deletions, substitutions and insertions of nucleotides.

In this aspect of the invention, the mutation may therefore be a mutation of any one of the genes explicitly identified above, that either changes the amino acid encoded by that portion of the gene or that does not change the encoded amino acid. A mutation may also code for an alternatively-spliced copy of the gene.

The invention also encompasses the use of DNA vectors that contain any of the above nucleotide sequences, optionally operatively-linked with one or more regulatory elements that direct expression of the coding sequences, in the manufacture of a medicament for the treatment or diagnosis of endometriosis. Genetically-engineered host cells that contain any of the above-described nucleotide sequences are also included in this aspect of the invention.

The present invention is further illustrated by the following examples that in no way should be construed as being limiting. The entire contents of all references cited throughout this application are expressly incorporated by reference.

### BRIEF DESCRIPTION OF THE FIGURES AND TABLES

FIG. 1 is a schematic illustration of the cleavage products of a restriction reaction.

FIG. 2 is a schematic illustration of DDRT-PCR-based expression profiling.

FIG. 3 shows the sequences of the primers used for RT-PCR validation experiments.

FIG. 4a presents RT-PCR data of SFRP4 using end point PCR. Consistent with the DDRT-PCR Indexing data, SFRP4 levels are higher in mRNA from healthy tissue of women A and B (Day 10 and 12 of the cycle). The reverse pattern is observed for women C and D (Day 2 and 3 of the cycle).

FIG. 4b (parts I and II) presents real time quantitative PCR data of SFRP4 using TaqMan technology. The X axis lists the patient samples examined, which are; patient G (Day 16), K (Day 6), I (Day 24), J (Day 8), P (Day 15), O (Day 3), L (on Zoladex) and N (Day 5). Y axis shows the Difference in Threshold Value After GAPDH Normalisation.

Threshold Value is defined as the cycle number that the fluorescent signal produced by the PCR becomes higher than the noise level and the PCR enters the log-linear phase. Difference in Threshold Value After GAPDH Normalisation is the GAPDH mRNA Threshold Value of a sample minus the sFRP4 mRNA Threshold Value.

The lower the Difference in Threshold Value after GAPDH Normalisation, the less mRNA transcript is present in the sample. For example, for patient K, KH>KUsl>KV1>KV2.

FIG. 5a presents RT-PCR data of immunoglobulin λ using end-point PCR. Consistent with DDRT-PCR Indexing data, immunoglobulin λ levels are higher in diseased tissue from patients A, B and D and almost absent in healthy tissues.

FIG. 5b compares the differences in gene expression between healthy and diseased endometrium tissues of individual patients as determined by end point PCR, with PCR products measured by densitometry. The Y axis reflects the densitometry value subscribed to the total intensity of each PCR band on an 1.3% agarose gel. The X axis lists the patients samples examined. It should be noted that comparisons should only be made within patients and not between group of patients.

FIG. 6a presents RT-PCR data of PAEP using end-point PCR. Consistent with the DDRT-PCR Indexing data, mRNA levels are higher in mRNA from healthy tissues of patients C and D. Additionally, patients A and B were found to have higher PAEP mRNA levels in healthy tissues. The double band for PAEP is derived from the different spliced forms of the PAEP gene.

FIG. 6b compares the differences in gene expression between healthy and diseased endometrium tissues of individual patients as determined by end point PCR, with the PCR products measured by densitometry. The Y axis reflects the densitometry value subscribed to the total intensity of each PCR band on a 1.3% agarose gel. The X axis lists the patients samples examined. It should be noted that comparisons should only be made within patients and not between group of patients.

FIG. 7 illustrates RT-PCR data of ferritin L chain (7a), cathepsin D (7a) stromelysin (7b), pro-alpha-1 type III collagen (7c) vitamin D3 25 hydroxylase (7d) and elongation factor-1 alpha (7e).

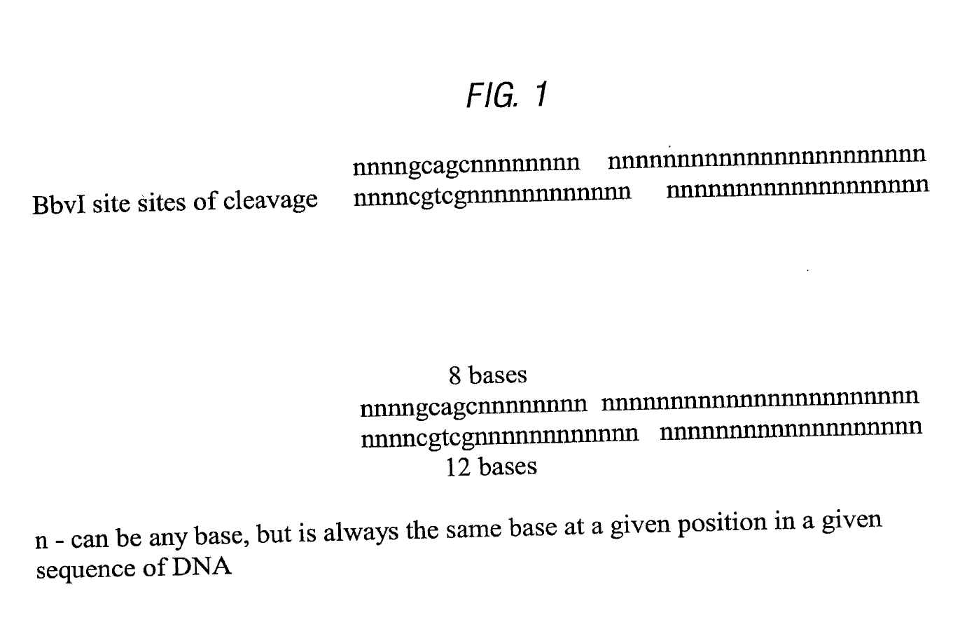

Table 1 includes the materials used for the DDRT-PCR study.

Table 2 includes the materials used for post-DDRT-PCR validation studies.

Table 3 lists the differentially expressed genes as determined by DDRT-PCR.

### EXAMPLES

### RNA Isolation

Human tissue samples were kindly donated by the Dept. of Obstetrics & Gynaecology, Univ. of Cambridge, Cambridge, UK and South Cleveland Hospital, Middlesborough, UK. Ethical approval was obtained for the study.

Initially, total RNA was isolated from matched ectopic and eutopic endometrium tissues from 4 women suffering from endometriosis A, B, C and D (see Table 1) and at a later stage of the study RNA was isolated from patients K, P and I (see Table 2). Ectopic endometrium was isolated from the ovaries (V), rectovaginal (R) and uterosacral (U) regions. Healthy endometrium is designated as H. The tissues were disrupted using a rotor stator-homogeniser and total RNA was isolated using the Rneasy Midi Kit (QIAGEN) according to the manufacturer's instructions. RNA yield was determined by measuring absorbency at 260 nm (Table 1 and 2) and RNA integrity was validated by non-denaturing agarose gel electrophoresis.

10 μg of RNA was loaded on a 1% non-denaturing agarose gel (Molecular Cloning; A Laboratory Manual, Sambrook, Fritsch & Maniatis, Cold Spring Harbor Laboratory Press) and the gel was run in 1×TBE buffer. The 18S and 28S ribosomal RNA bands were visualised by EtBr staining (0.51 μg/ml). No high molecular weight bands were observed confirming the absence of genomic DNA.

TABLE 1
Material used for endometriosis study.

| Healthy | | Total | No. of possible |
|---|---|---|---|
| Endometrium | Menstrual Cycle | RNA (μg) | adaptor pool pairs |
| A/H | Day 10 | 304 | 136 |
| B/H | Day 12 | 441 | 136 |
| C/H | Day 4 | 290 | 136 |
| D/H | Day 3 | 26 | 26 |

| | | No. of | | | No. of |
|---|---|---|---|---|---|
| | Total | possible | | Total | possible |
| Ovarian | RNA | adaptor | Recto-vaginal | RNA | adaptor |
| endometriosis | (μg | pool pairs | endometriosis | (μg | pool pairs |
| A/V | 12 | 12 | — | — | — |
| B/V | 40 | 40 | B/R | 0 | 0 |
| C/V | 2 | 2 | C/R | 9 | 9 |
| D/V | 64 | 64 | D/R | 2 | 2 |

### RNA Validation

All RNA samples were validated for the absence of genomic DNA and presence of long transcripts by RT-PCR using β-actin, VEGF and fit specific primers.

First strand cDNA was synthesised from 1-5 μg of total RNA using M-MLV reverse transcriptase and oligo (dT)₁₂₋₁₈ primer (SuperScript Choice System for cDNA synthesis; Gibco BRL). 1 ng of single strand cDNA was amplified in 10 μl reaction containing 0.5 μM primer, 4×0.2 mM dNTPs, 1×AmpliTaq Gold PCR buffer and 2.5U of AmpliTaq Gold DNA Polymerase (Perkin Elmer). Hot Start PCR was performed as follows; 1 cycle of 95° C. for 12 min; 35 cycles of 95° C. for 30 sec, 55° C. for 30 sec and 72° C. for 1 min 30 sec; 1 cycle of 72° C. for 10 min. The annealing temperature was 55° C. for β-actin and VEGF primers while annealing was performed at 50° C. for flt11 and 19 primer pair and 60° C. for flt13 and 21 primer pair. The β-actin and human VEGF primer sets were supplied by R&D Systems. The fit primers 11 and 19 span regions 641-660 and 1524-1542 of human fit receptor while the fit primers 13 and 21 span regions 1362-1382 and 2138-2152 respectively. All 10 μl of each PCR reaction were loaded on 1% agarose gel.

### Preparation of EDNA

Double stranded cDNA was synthesised from the whole total RNA preparation (up to 110μg) using oligo(dT)₁₂₋₁₈ primer and in the presence or absence (for negative control) of reverse transcriptase SuperScript II (SuperScript II Choice System; Gibco BRL) according to manufacturer's protocol. The cDNA was then purified by using the microcentrifuge purification protocol of the QIAquick PCR Purification Kit (QIAGEN). To confirm synthesis of full length cDNA as well as absence of genomic DNA, the double stranded cDNA was validated in the same manner as the first strand cDNA described in the RNA validation section.

### Indexing DDRT-PCR

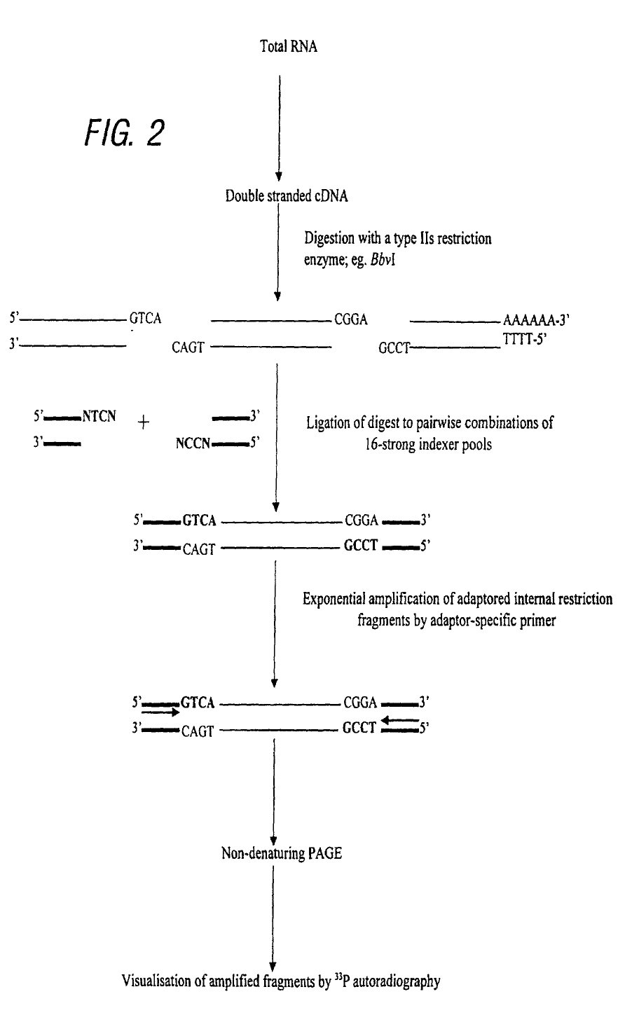

Indexing DDRT-PCR was performed as described by Mahadeva et al. (J.Mol.Biol. (1998) 284;1391-1398). A summary depiction of the method is provided in FIG. 2 herein. The method involves the following steps;

Restriction digestion

15 μg for 10 adaptor pools of total RNA was digested with 12U of BbvI (New England BioLabs), in a total volume of 120 μl containing 12 μl of 10×BbvI buffer. The digestion was carried out at 37° C., for 3 hrs. The restriction enzyme was inactivated by incubation of the reactions at 65° C. for 10 min. The digested cDNA was purified using the microcentrifuge protocol of the QIAquick PCR Purification Kit (QIAGEN).

Ligation reactions

The Sac adaptor pools used were as described by Mahadeva et al. (J.Mol.Biol. (1998) 284;1391-1398) and had the overhangs shown in Table 2. 0.25 μg of total RNA cDNA equivalents were ligated in triplicate to 10 different pairwise combinations of adaptor pools (Table 2). Each ligation reaction comprised 2 nM of each adaptor pool, 0.6 mM ATP, 1×T4 ligase buffer in a volume of 25 μl. An additional 25 μl of 0.5U/μl of T4 DNA ligase (Gibco BRL) were added after heating the ligation mixtures to 65° C. for 5 min and cooling at 37° C. for 2 min in a PCR machine. After addition of the ligase, ligations proceeded for 37° C. for 1 hr. The T4 DNA ligase was inactivated by incubation at 65° C. for 10 min. Unincorporated adaptors were removed by using the vacuum manifold protocol of the QIAquick PCR Purification Kit (QIAGEN).

Isolation of differentially expressed genes

2.5 μl of each adaptored cDNA were amplified in a 10 μl reaction containing 0.5 μM Sac primer with sequence 5′-TAGCACGACTCAGAGCTCAT-3′, 4×O.2 mM dNTPs, 33 nM 1000-3000 Ci/mmol [33P]dATP (Amersham Pharmacia Biotech), 1×AmpliTaq Gold buffer and 2.5U of AmpliTaq Gold (Perkin Elmer). The PCR conditions were; 1 cycle, 95° C., 10 min; 30 cycles of 95° C, 30 s; 60° C, 30 s; 72° C., 1.5 min; 1cycle, 72° C., 10 min. Amplified cDNA fragments were size separated through a 4.8% (w/v) non-denaturing polyacrylamide gel. The 3 “+RT cDNA” ligation reactions and the one “−RT cDNA” ligation reaction from healthy tissue were loaded side by side to the ones from the diseased tissues for comparison. 10 μl of 33P-labelled PCR product were mixed with 1 μl of 33P-labelled “Gel loading standard” and 3.25 μl of 4×non-denaturing loading dye. The loading dye consisted of 20% (v/v) glycerol, 0.2% (w/v) bromophenol blue, 0.2% (w/v) xylene cyanol, 10 mM EDTA (pH8.0). The “Gel loading standard” was used for establishing and correcting the accuracy of sample loading onto the gel. This standard is a 1126 bp fragment of lambda DNA made by PCR using primers lambda-Pr1 and lambda-Pr2 at the conditions described for the adaptored cDNA in a total volume of 80 μl and at an annealing temperature of 60° C. Gels were visualised by exposure on Phospho Screen (Molecular Dynamics) at room temperature, overnight as well as autoradiography for approximately a week.

Bands representing putative differentially expressed cDNAs were categorised as follows:

I=fragment represents a difference between the healthy and “diseased” endometrium of an individual, and the difference is seen in one or more other individuals suffering of endometriosis in the same region of the reproductive tract;

II=fragment represents a difference between the healthy and “diseased” endometrium of an individual, and the difference is seen in one or more other individuals suffering of endometriosis in different regions of the reproductive tract;

III=fragment represents a difference between the healthy and “diseased” endometrium of an individual.

The putatively differentially expressed cDNA fragments were isolated using the method described by Reeves et al. (BioTechniques, 1995, 18, 18-20), reamplified using the Sac primer in a volume of 30 μl and the reamplified products were purified by PEG precipitation (Rosental et al., 1993, NAR 21, No. 1, 173-174). The sizes of the Sac adaptored PCR fragments are listed in Table 3. Once purified, the cDNA fragments were subcloned in pGEM vector using the pGEM-T Easy Vector System II (Promega) according to manufacturer's protocols. The cloned inserts were amplified by using vector primers M13F and M13R from 5 individual clones per adaptor pool and the PCR products were purified by PEG precipitation.

### Data Analysis

Differentially expressed cDNA fragments were sequenced by the dye terminator cycle DNA sequencing method, using the DNA Sequencing Kit from ABI Prism in a 377 sequencer (Perkin Elmer). The chromatograms were analysed using the ABIPRISM DNA Sequencing Analysis Software (Perkin Elmer) and the sequence data were screened for similarity against the entire public nucleic acid and protein databases (BLAST).

### Validation of Differential Gene Expression Results by RT-PCR

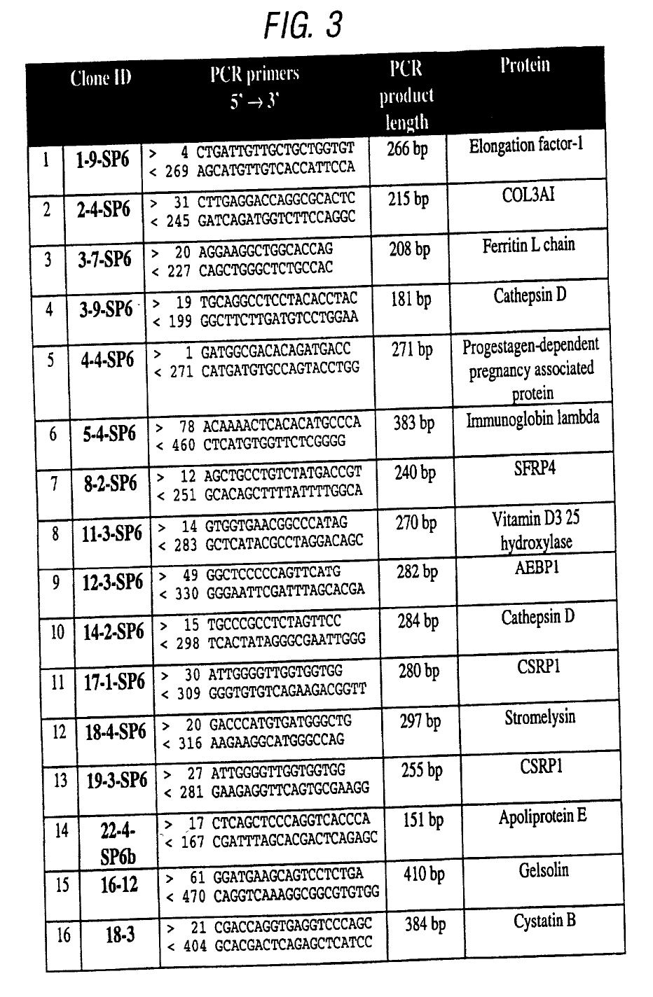

Primers specific for the genes to be validated were designed (see FIG. 3) and were used in an RT-PCR experiment as follows. 0.1-1 ng of single stranded cDNA was used and amplifications were performed in a total volume of 10 μl using 0.5 μM primer, 4×0.2 mM dNTPs, 1×AmpliTaq Gold PCR buffer and 2.5U of AmpliTaq Gold DNA Polymerase (Perkin Elmer). Hot Start PCR was performed as follows; 1 cycle of 95° C. for 10 min; 20-30 cycles of 95° C. for 30 sec, 59° C. for 30 sec and 72° C. for 1.5 min; 1 cycle min. The whole PCR reaction was loaded on 1.8% agarose gel and bands were visualised by EtBr staining.

RT-PCR experiments with a larger number of patients listed in Table 2 were performed as follows; 0.01 μg of total RNA were reverse transcribed to produce single stranded cDNA using Superscript II and amplifications were performed in a total volume of 25 μl containing 0.5 μl of diluted cDNA, 12.5 μl of HotStarTaq Master Mix (QIAGEN) containing HotStarTaq polymerase, 1 μl of each gene specific primer (10 pmol/μl) and 10 μl of water. Three dilutions of cDNA were used as template, 1:10, 1:100 and 1:1000. Amplification was performed as follows; 1 cycle of 95° C. for 15min; 35 cycles of 94° C. for 30 sec, 60° C. for 30 sec and 72° C. for 2 min; 1 cycle of 72° C. for 10 min. 10 μl of the PCR reaction was loaded on 1.2% agarose gel and bands were visualised by EtBr staining.

TABLE 2
Material used for validation of the genes discovered to be
differentially expressed in endometriosis.

| | Day of | | Total |
|---|---|---|---|
| | Menstrual | | RNA |
| Patient | Cycle | Pathology | (μg) |
| O | 3 | Healthy (O/H) | 0.592 |
| | | Ovarian Left endometriosis (O/V) | 0.840 |
| N | 5 | Healthy (N/H) | 70 |
| | | Ovarian Right endometriosis (N/V) | 8.3 |
| K | 6 | Healthy (K/H) | 309.0 |
| | | Uterosacral (K/Usl) | 18.4 |
| | | Ovarian endometriosis (K/V1) | 2.39 |
| J | 8 | Healthy (J/H) | 55.0 |
| | | Uterine fundal endometriosis (K/UF) | 0.28 |
| P | 15 | Healthy (P/H) | 0.28 |
| | | Ovarian Right endometriosis (P/Vr) | 0.18 |
| G | 16 | Healthy (G/H) | 56.5 |
| | | Ovarian endometriosis (G/V) | 2.57 |
| I | 24 | Healthy (I/H) | 0.314 |
| | | Ovarian Left endometriosis (I/V1) | 0.058 |
| L | Non menstrual. | Healthy (L/H) | 2.0 |
| | On Zoladex. | Rectovaginal endometriosis (L/RV) | 2.8 |
| | | Rectal endometriosis (L/R) | 3.0 |

### Results

The cDNA populations produced from the samples described on Table 1 were sorted into distinct subsets by using adaptor pools A-O. The identities of the differentially expressed bands are reported in Table 3.

TABLE 3
Summary - analysis of differentially expressed DNA bands

| | | | | | 98% (²¹⁴/_{217b}) ID. to |
|---|---|---|---|---|---|
| | | | | | Human ferritin L chain |
| | | | 3-9 | | M11147 |
| | | | | | 207-423 |
| | | I | 3-7 | | 99% (²²⁸/_{230b}) ID. to |
| Pool C | 3 | +BV, −BH | 3-4 | 262 | Human cathepsin D from oestrogen |
| 16 + 11 | | +DV, −DH | 3-8 | | responsive breast cancer cells |
| | | | 3-10 | | M11233 |
| | | | 3-11 | | 1740-1969 |
| | | | 3-12 | | |
| | | | 3-13 | | |
| | | | 3-14 | | |
| | | | 3-15 | | |
| | | | 3-16 | | |
| | | II | 49-1 | | 83% (⁴¹⁴/_{497b}) ID. to |
| Pool M | 48 | +KU, −KH | 49-4 | 342 | Human ferritin L chain |
| 8 + 12 | | +BV, −BH | 49-6 | | M11147 |
| | | | | | 443-640 |
| | | II | 23-1 | | |
| | | CR > CH, | 23-2 | | 95% (⁴⁰⁰/_{420b}) ID. to |
| Pool A1 | 23 | AV > AH, | 23-3 | 501 | Human ferritin H chain |
| 4 + 16 | | BV > BH | 23-4 | | M11146 |
| | | | 23-5 | | 281-697 |
| | | | 23-6 | | |
| | | I | 12-1 | | |
| | | +AV, −AH | 12-5 | | 98% (²⁹⁹/_{304b}) ID. to |
| Pool H | 12 | +BV, −BH | 12-3 | 320 | AE-binding protein 1 (AEBP1) |
| 7 + 5 | | +DV, −DH | 12-10 | | D86479 |
| | | | 12-13 | | 657-956 |
| | | | 12-16 | | |
| | | I | 14-2 | | |
| Pool D | | +AV, −AH | 14-3 | | 99% (²²⁸/_{230b}) ID. to |
| 9 + 11 | 14 | +BV, −BH | 14-4 | 243 | Human cathepsin D |
| | | +DV, −DH | 14-9 | | M11233 |
| | | | 14-10 | | |
| | | | 14-12 | | |
| | | | 14-13 | | |
| | | | 14-14 | | |
| | | I | 18-3 | | |
| | | +DV, −DH | 18-9 | | |
| Pool D | 18 | +AH, −AV | | 400 | 96% (²⁶⁹/_{280b}) ID to |
| 9 + 11 | | +BH, −BV | | | Cystatin B |
| | | | | | AF208234 |
| | | II | 18-6 | | |
| Pool D | 18 | +DV, −DH | 18-2 | 400 | 97% (³⁸²/_{390b}) ID. to |
| 9 + 11 | | +AH, −AV | 18-4 | | Human Stromelvsin-3 |
| | | +BH, −BV | 18-12 | | X57766 |
| | | +CH, −CR | 18-14 | | 240-628 |
| | | II | 25-1 | | |
| Pool C | 25 | DV > DH | 25-2 | 471 | 98% (³⁸⁴/_{390b}) ID. to |
| 11 + 16 | | CH > CR | 25-3 | | Human Stromelysin-3 |
| | | AH > AV | 25-5 | | NM_005940 |
| | | BH > BV | 25-6 | | 240-628 |
| | | | 25-7 | | |
| | | I | 37-3 | | |
| Pool I | 37 | DV > DH | 37-5 | 372 | 98% (²⁷⁵/_{279b}) ID to |
| 7 + 15 | | AH > AV | | | Human Stromelvsin-3 |
| | | BH > BV | | | NM_005940 |
| | | | | | 691-969 |
| | | II | 31-2 | | 98% (³²⁰/_{324b}) ID. to |
| Pool F | 31 | +DH, −DV | 31-4 | 415 | Protease inhibitor 1 (PI 1) |
| 4 + 9 | | +CH, −CR | 31-5 | | X01683 |
| | | | 31-7 | | 814-1145 |
| | | | 31-8 | | |
| | | | 8-2 | | |
| Pool E | | I | 8-5 | | 99% (²⁵⁰/_{252b}) ID. to |
| 9 + 10 | 8 | +AH, −AV | 8-1 | 296 | Human secreted frizzled-related protein |
| | | +BH, −BV | 8-4 | | 4 (SFRP4) |
| | | | 8-10 | | AF026692 |
| | | | 8-11 | | 626-877 |
| | | | 8-12 | | |
| | | | 8-15 | | |
| | | II | 16-1 | | 97% (^{∘}/_{617b}) ID. to |
| Pool D | 16 | +AV, −AH | 16-12S | 644 | Gelsolin |
| 9 + 11 | | +CR, −CH | 16-15 | | NM_000177 |
| | | | | | 459-1074 |
| | | II | 4-4 | | 93.9% (³⁵⁶/_{379b}) ID. to |
| Pool A | | +CH, −CV, −CR | 4-7 | | Human progestagen-dependent |
| 4 + 16 | 4 | +DH, −DV, −DR | 4-2 | 395 | pregnancy-associated endometrial |
| | | | 4-8 | | protein 14 |
| | | | 4-1 | | J04129 |
| | | | | | 370-716 |
| | | II | 26-1 | | 95% (¹⁶⁰/_{168b}) ID. to |
| Pool D | 26 | DH > DV | 26-3 | | Human progestagen-dependent |
| 9 + 11 | | +CH −CR | 26-5 | 396 | pregnancy-associated endometrial |
| | | | 26-6 | | protein 14 (PAEP) |
| | | | | | J04129 |
| | | | | | 49-337 |
| | | II | 59-1 | | 99% (³⁴⁹/_{351b}) ID. to |
| Pool A1 | 59 | DH > DV | 59-3 | | Human progestagen-dependent |
| 4 + 16 | | CH > CR | 59-4 | 429 | pregnancy-associated endometrial |
| | | AH > AV | 59-5 | | protein 14 (PAEP) |
| | | | | | J04129 |
| | | | | | 365-715 |
| | | I | 5-6 | | 98% (⁴⁵⁰/_{456b}) ID. to |
| Pool G | 5 | +AV, −AH | 5-5 | 495 | Human Immunoglobin lambda heavy |
| 4 + 9 | | +BV, −BR | 5-4 | | chain |
| | | +DV, −DH | 5-10 | | Y14737 |
| | | | 5-11 | | 741-1195 |
| | | | 5-12 | | |
| | | | 5-13 | | |
| | | | 5-14 | | |
| | | | 5-15 | | |
| | | I | | 5 | 82% (³⁶⁴/_{443b}) ID. to |
| | | +DV, −DR | | | Human complement component 3 (C3) |
| Pool N | | NV > NH | 44-2 | 44 | NM_000064 |
| 14 + 16 | 44 | +BV, −BH | 44-3 | | 4328-4770 |
| | | +PV, −PH | 44-4 | | |
| | | IV > IH | | | |
| | | II | 27-3 | | 96% (⁴⁴⁷/_{464b}) ID. to |
| Pool D | 27 | +DH, −DV | 27-4 | 753 | Insulin-like growth factor-binding |
| 9 + 11 | | +CH, −CR | 27-5 | | protein-3 (IGPBP-3) |
| | | | 27-6 | | M35878 |
| | | | | | 9365-9895 |
| | | II | 60-2 | | 97% (⁶⁰⁸_{625b}) ID. to |
| Pool C | 60 | +DH, −DV | 60-3 | 641 | Insulin-like growth factor-binding |
| 11 + 16 | | CH > CR | 60-4 | | protein-3 (IGFBP-3) |
| | | | 60-5 | | M35878 |
| | | | 60-6 | | 9429-10006 |
| | | | 60-7 | | |
| | | I | 43-1 | | 88% (⁴³¹/_{486b}) ID. to |
| Pool M | 43 | DV > DH | 43-2 | | Human dual specificity pbosphatase I |
| 8 + 12 | | +NV, −NH | 43-3 | 577 | NM_004417 |
| | | +KU, −KH | 43-4 | | 669-1154 |
| | | +BV, −BR | 43-6 | | |
| | | +PV, −PH | | | |
| | | I | 1-9 | | |
| Pool D | | AH > AV, | 1-10 | | 97.1% (³²⁹/_{339b}) ID. to |
| 9 + 11 | 1 | BH > BV | 1-3 | 384 | Human elongation factor 1 alpha |
| | | DH > DV | 1-6 | | subunit |
| | | | 1-7 | | X03558 |
| | | | | | 390-723 |
| | | I | 24-1 | | 99% (³⁵²/_{334b}) ID. to |
| Pool C | 24 | DH > DV | 24-3 | | Human elongation factor 1 alpha |
| 11 + 16 | | AH > AV | 24-4 | 409 | subunit |
| | | BH > BV | 24-5 | | X03558 |
| | | | 24-6 | | 764-1097 |
| | | | | | 110-776 |
| | | II | 55-1 | | 96% (²²⁵/_{232b}) ID. to |
| Pool H | 55 | +AV, −AH | 55-2 | | Nascent-polypeptide-associated |
| 5 + 7 | | BV > BH | 55-3 | 278 | complex alpha polypeptide (NACA) |
| | | CR > CH | 55-4 | | AF054187 |
| | | | 55-5 | | 485-716 |
| | | | 55-6 | | |
| | | II | 2-10 | | |
| Pool D | | +AH, −AV | 2-4 | | 93.9% (⁵⁰⁸/_{541b}) ID. to |
| 9 + 11 | 2 | +BH, −BV | 2-7 | 594 | Human pro-alpha-1 type III collagen |
| | | +CH, −CR | 2-3 | | x14420 |
| | | DH < DV | 2-5 | | 3150-3395 |
| | | | | | 2866-3120 |
| | | II | 33-1 | | 93% (²⁷⁸/_{298b}) ID. to |
| Pool E | 33 | +AH, −AV | 33-5 | 675 | Human collagen alpha 1 type III |
| 9 + 10 | | +BH, −BV | 33-6 | | (COL3A1) |
| | | +CH, −CR | 33-7 | | NM_000090 |
| | | | 33-8 | | 2858-3391 |
| | | II | 33-1 | | 97% (²⁷⁶/₂₇₃) ID to |
| Pool E | 34 | +AH, −AV | 33-5 | 708 | Human collagen alpha 1 type III |
| 9 + 10 | | +BH, −BV | 33-6 | | (COL3A1) |
| | | +CH, −CR | 33-7 | | NM_000090 |
| | | | 33-8 | | 1908-2218 |
| | | | | | 1651-1968 |
| | | II | 36-1 | | 95% (⁵¹⁶/_{542b}) ID to |
| Pool J | 36 | +CH, −CR | 36-2 | 675 | Human collagen alpha 1 type III |
| 9 + 13 | | AH > AV | 36-3 | | (COL3A1) |
| | | BH > BV | 36-4 | | NM_000090 |
| | | | 36-5 | | 2858-3398 |
| | | | 36-7 | | |
| | | I | 39-1 | | 85% (⁴⁶²/₅₃₉) ID to |
| Pool K | 39 | KH > KU | 39-3 | 909 | Human collagen type I alpha 2 |
| 3 + 12 | | BH > BV | 39-4 | | (COL1A2) |
| | | DV > DH | 39-5 | | NM_000089 |
| | | IV > IH | 39-6 | | 1160-1924 |
| | | | 39-7 | | |
| | | II | 48-2 | | 86% (²⁵⁰/_{297b}) ID. to |
| Pool M | 48 | +NH, −NV | 48-3 | 353 | Human procollagen C-endopeptidase |
| 8 + 12 | | +KH, −KU | 48-4 | | enhancer |
| | | +BH, −BV | 48-6 | | NM_002593 |
| | | +IH, −IV | | | 1167-1291 |
| | | PV > PH | | | |
| | | II | 62-1 | | 95% (³⁸⁰/_{415b}) ID to |
| Pool G | 62 | DH > DV | 62-3 | 462 | Human proline 4-hydroxylase beta |
| 5 + 15 | | CH > CR | 62-5 | | polypeptide |
| | | AH > AV | 62-6 | | NM_000918 |
| | | BH > BV | | | 1150-1567 |
| | | II | 63-1 | | 94% (²¹¹/_{224b}) ID. to |
| Pool E | | +DH, −DV | 63-2 | 270 | Claudin 4 (CLDN4) |
| 9 + 10 | 63 | +CH, −CR | 63-3 | | NM_001305 |
| | | | 63-5 | | 471-692 |
| | | | 63-6 | | |
| | | | 63-7 | | |
| | | II | 47-2 | | 81% (²⁷⁰/_{330b}) ID. to |
| Pool O | 47 | −KH, +KU | 47-3 | 455 | Human melanoma adhesion protein |
| 8 + 13 | | −PH, +PV | 47-4 | | (MCAM) |
| | | −IH, +IV | 47-5 | | NM_906500 |
| | | | | | 1965-2315 |
| | | I | 11-1 | | |
| Pool H | | +BV, −BH | 11-4 | | 100% (²⁷⁷/_{277b}) ID. to |
| 7 + 5 | 11 | +DV, −DH | 11-2 | 302 | Vitamin D3 25 hydroxylase |
| | | | 11-5 | | X59812 |
| | | | 11-3 | | |
| | | II | | | 97% (³²⁶/_{355b}) ID. to |
| Pool D | 17 | +AV, −AH | 17-1 | 388 | Human cystein & glycine-rich protein |
| 9 + 11 | | +CR, −CH | 17-7 | | 1 (CSRP1) |
| | | | 17-16 | | M33146 |
| | | | | | 47-379 |
| | | II | | | 99% (³⁶¹/_{362b}) ID. to |
| Pool C | 19 | +AV, −AB | 19-15 | 380 | Human cystein & glycine-rich protein |
| 16 + 11 | | +BV, −BH | 19-3 | | 1 (CSRP1) |
| | | +CR, −GB | 19-2 | | M33146 |
| | | | | | 15-376 |
| | | I | 32-1 | | 93% (¹⁸⁹/_{203b}) ID. to |
| Pool G | 32 | DV > DH | 32-2 | 285 | Prosaposin (SAP1) |
| 5 + 15 | | AV > AH | 32-3 | | J03015 |
| | | BV > BH | 32-4 | | 1545-1761 |
| | | | 32-5 | | |
| | | | 32-6 | | |
| | | I | | | 85% (³²¹/₃₇₆) ID. to |
| | | −DH, +DV | 45-6 | | Human steroidgenetic acute reaulatory |
| Pool N | 45 | −NH, +NV | 45-7 | 505 | protein (STAR) |
| 14 + 16 | | −BH, +BV | 45-8 | | NM_000349 |
| | | −PH, +PV | 45-9 | | 1004-1378 |
| | | −IH, +IV | 45-10 | | |
| | | I | 22-10 | | 99% (¹³⁸/_{139b}) ID. to |
| Pool B | 22 | +BV, −BH | 22-11 | 177 | Human Apoliprotein E |
| 4 + 2 | | +DV, −DH | 22-4 | | X00199 |
| | | | 22-2 | | 3-141 |
| | | II | | | 99% (²¹⁵/_{217b}) ID. to |
| Pool B | 50 | DV > DH | 50-2 | | Human Transcobalamin II |
| 2 + 4 | | CR > CH | 50-3 | 270 | NM_000355 |
| | | AV > AH | 50-4 | | 1022-1238 |
| | | BV > BH | | | |
| | | I | | | 99% (²⁶⁷/_{268b}) ID. to |
| Pool E | 54 | −NH, +AV | 54-1 | 318 | Human early growth response 1 |
| 9 + 10 | | BV > BH | 54-2 | | (EGR1) |
| | | | | | NM_001964 |
| | | | | | 565-832 |
| | | II | 40-1 | | 79% (³⁴²/_{431b}) ID. to |
| Pool K | 40 | BH > DV | 40-2 | 730 | Human podocalyxin |
| 3 + 12 | | KH > KU | 40-3 | | NM_005397 |
| | | IH > IV | 40-4 | | 2201-2631 |
| | | | 40-5 | | |
| | | | 40-6 | | |
| | | II | 41-1 | | 83% (⁴¹⁴/_{497b}) ID. to |
| Pool K | 41 | KH > KU | 41-2 | 634 | RAD21 |
| 3 + 12 | | BH > BV | 41-3 | | NM_006265 |
| | | DH > DV | 41-5 | | 965-1461 |
| | | IH > IV | 41-6 | | |
| | | II | 52-1 | | 100% (²⁵⁰/_{253b}) ID. to |
| Pool B | 52 | DH > DV | 52-2 | 295 | Human guanine nucleotide binding |
| 2 + 4 | | CH < CR | 52-3 | | protein beta polypeptide 2-like 1 |
| | | | 52-6 | | (RACK1) |
| | | | | | NM_006098 |
| | | | | | 595-834 |
| | | I | 53-1 | | 90% (³⁸⁰/_{420b}) ID. to |
| Pool D | 53 | AH > AV | 53-3 | 528 | Human ribosomal protein S6 |
| 9 + 11 | | BH > BV | 53-4 | | NM_001010 |
| | | | 53-5 | | 179-597 |
| | | II | 58-2 | | 92% (⁴⁶⁵/_{505b}) ID. to |
| Pool O | 58 | NH > NV | 58-3 | 544 | Human adenosine deaminase RNA |
| 8 + 13 | | KH > KU | 58-4 | | specific |
| | | PH > PV | 58-5 | | NM_015841 |
| | | | 58-6 | | 5864-6373 |

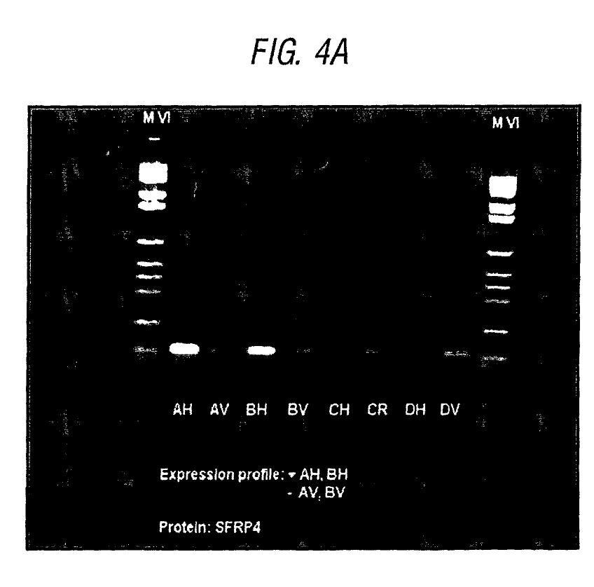

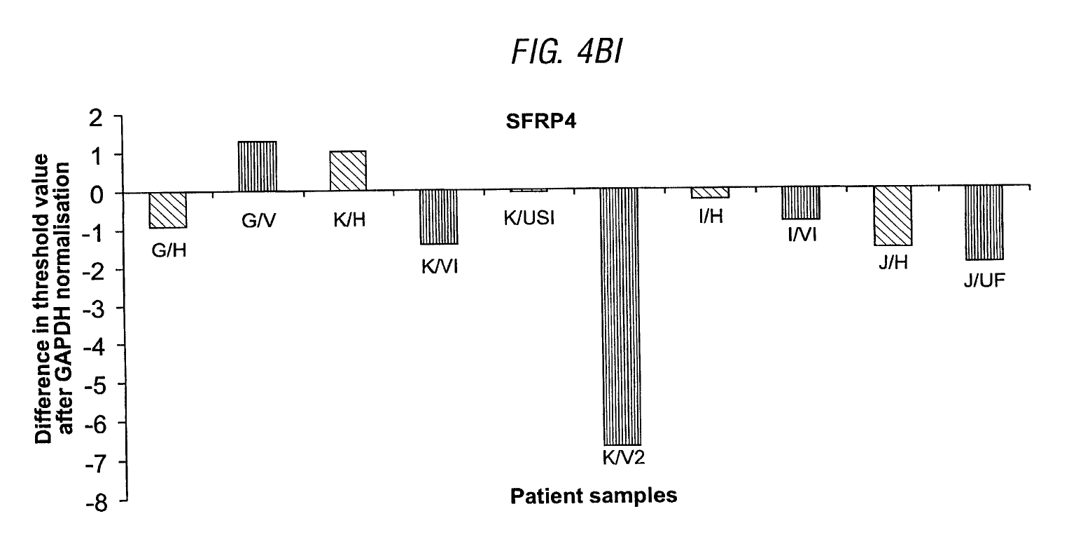

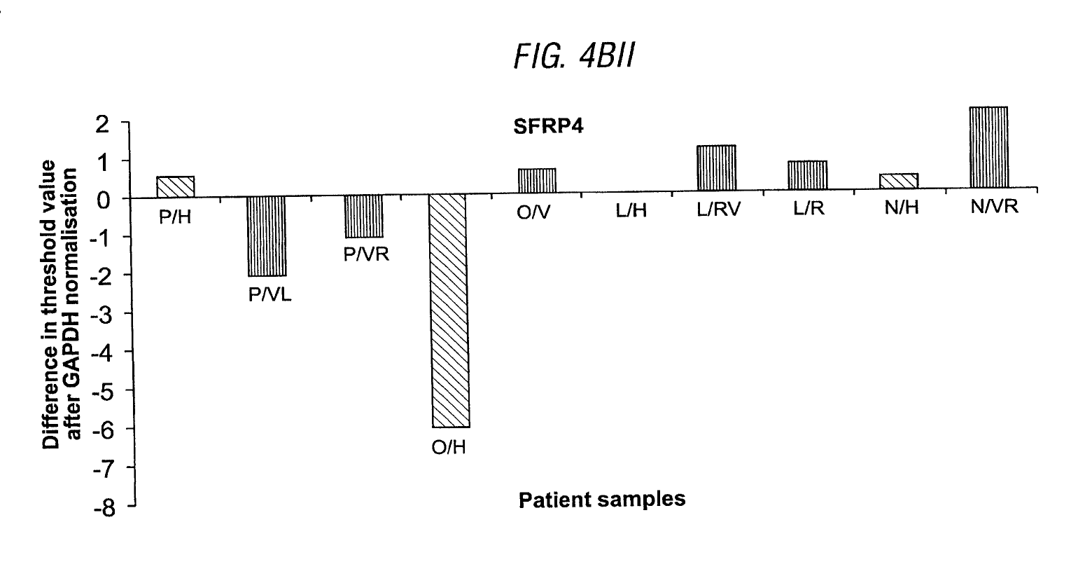

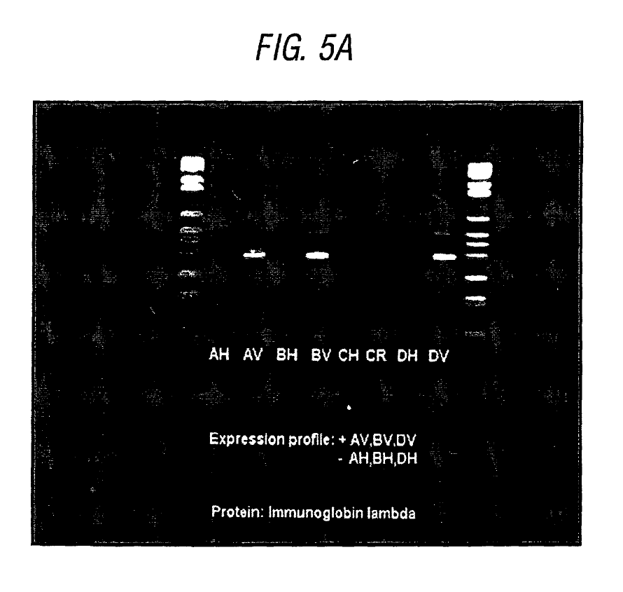

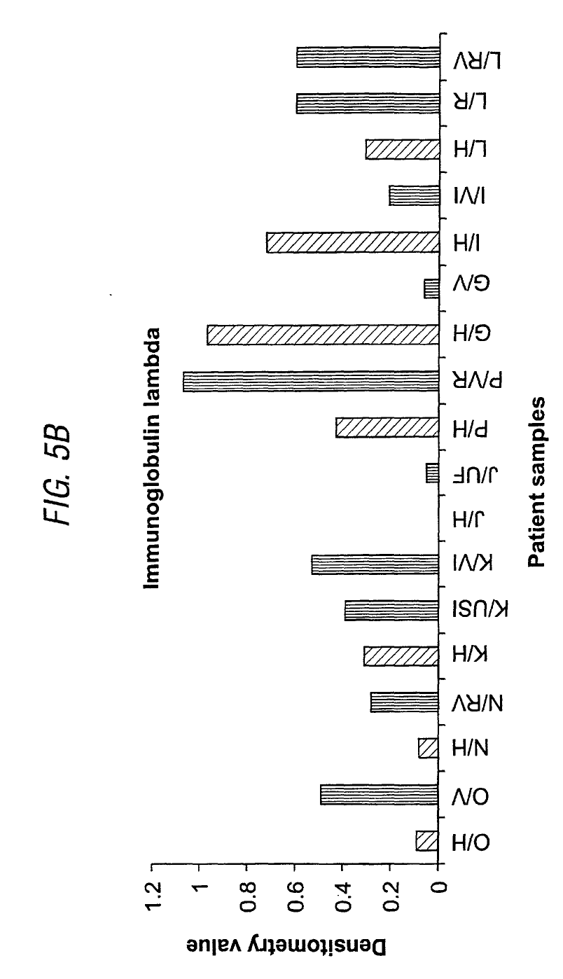

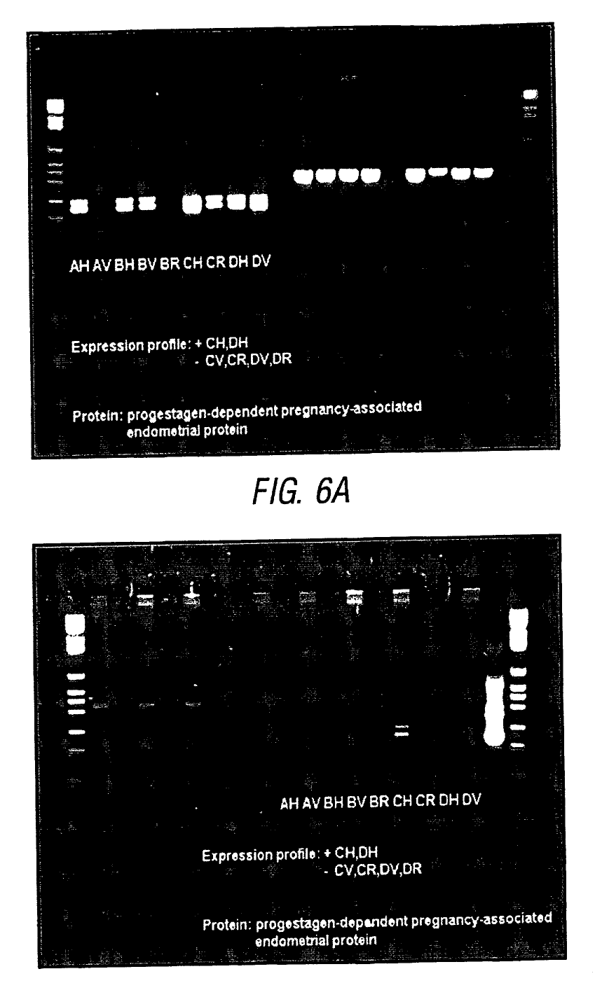

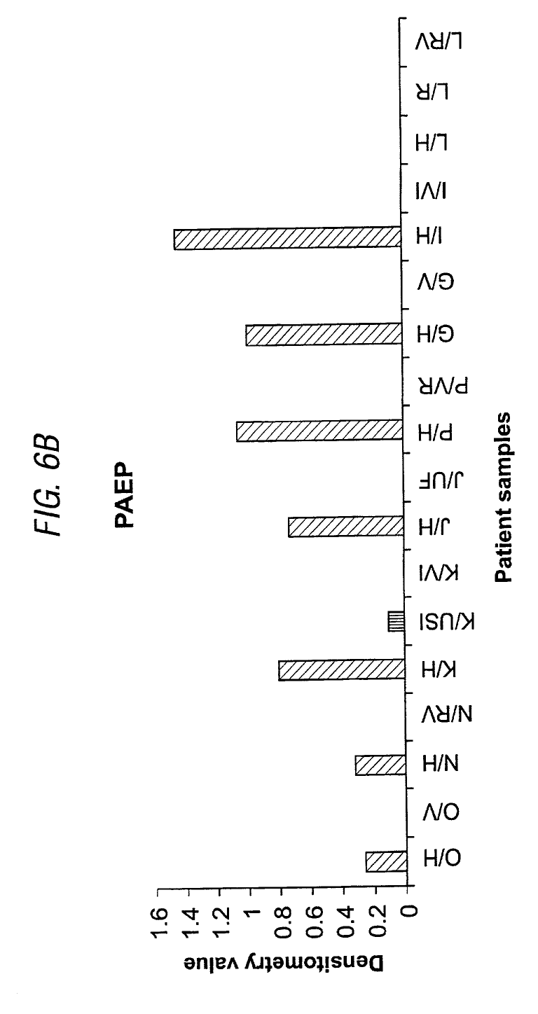

The majority of the DDRT-PCR data were validated and confirmed by an independent method, RT-PCR, using primers specific for each discovered gene. Validation by RT-PCR was performed in all patient samples shown in Tables 1 and 2. The validation data for PAEP, immunoglobulin λ and sFRP-4 are shown in FIGS. 4 and 5.

A detailed description of each identified protein is given below. The literature contains multiple names for some of these proteins. The accession numbers should be considered the unique descriptor.

### PROTEASES AND PROTEASE INHIBITORS

Cathensin D

Cathepsin-D is an aspartyl protease that has been implicated in increased risk of metastasis in breast and other cancers. Cathepsin D facilitates cell growth and invasion by digesting basement membranes, extracellular matrix proteoglycans and connective tissue. Cathepsin D displays an autocrine activity and is mitogenic after autoactivation at acidic pH facilitating invasion. It now appears that cathepsin D may play a similar role in endometriosis.

A comparison of cathepsin D levels in endometriotic tissue and in uterine endometrium has been published by Bergqvist et al. (1996, Fertis. Steril., 65(6): 1130-1134). Samples of endometriotic tissue and uterine endometrium were obtained from the same women and cathepsin D levels were determined by an immunoradiometric method. The cathepsin D level was significantly higher in endometriotic tissue at ectopic sites than in eutopic endometrium in both the proliferative (˜Days 1-14) and secretory (˜Days 14-28) phase of the cycle. In contrast to the findings of Garcia et al. (J. Steroid Biochem., (1987), 27: 439-445), cathepsin D mRNA was found in normal endometrium. Berqvist et al. (1996) explain their contradictory data by claiming the high sensitivity of their method rather than the less sensitive immunostaining results obtained by Garcia et al.

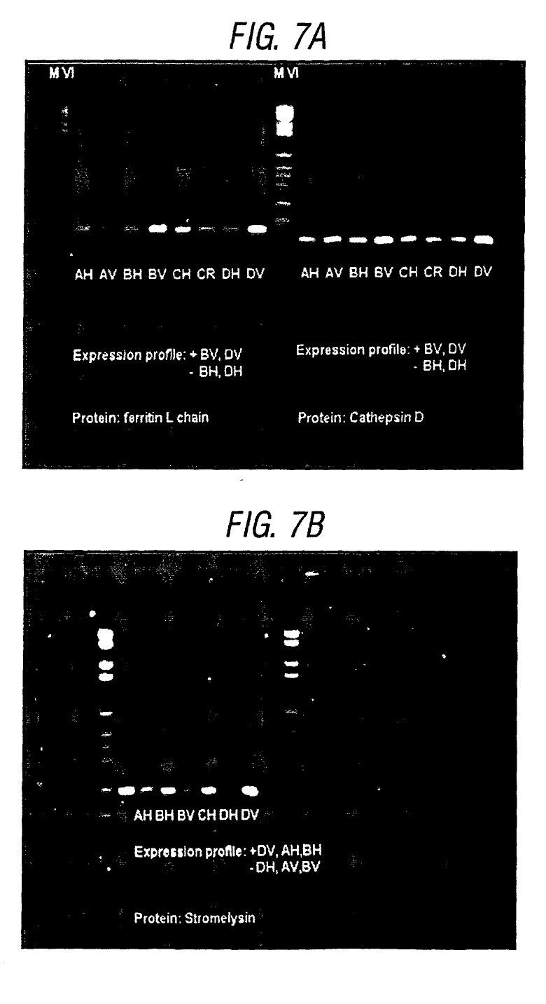

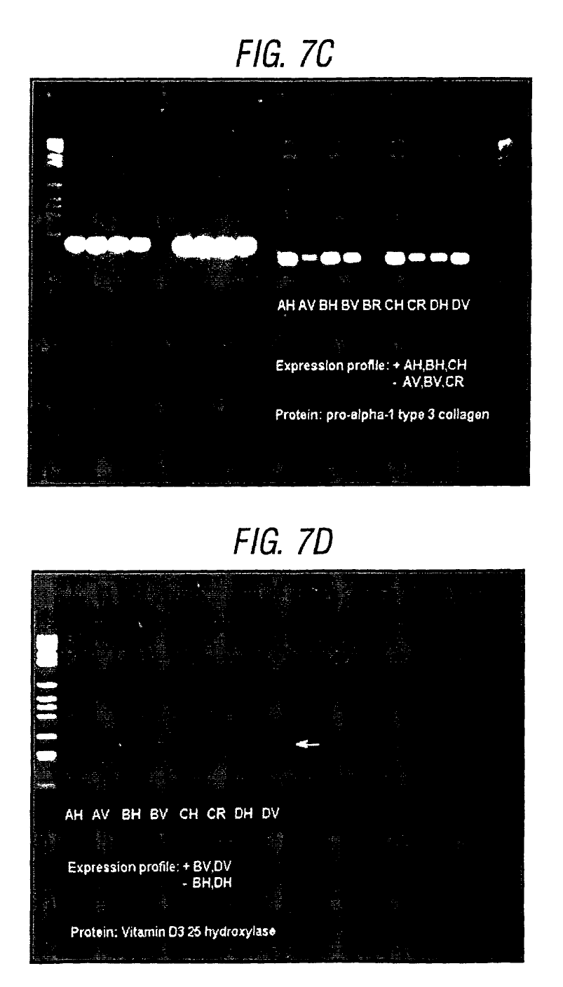

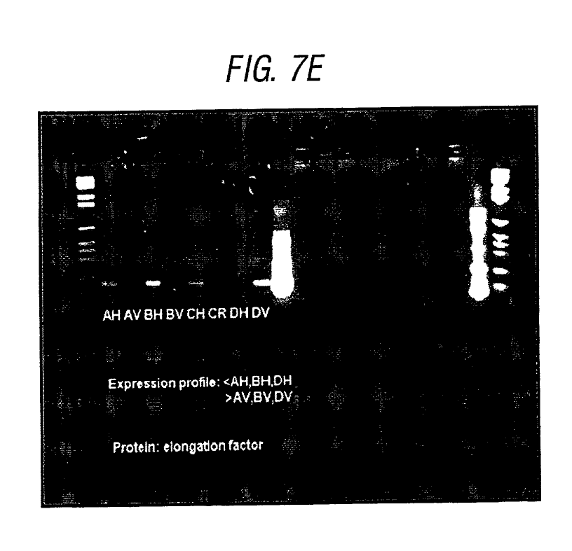

Interestingly, differentially expressed cathepsin D gene fragments are reported herein through two different adaptor pools (Pool C and D in Table 1). These two fragments represent sequences 1335-1564 of the human cathepsin D gene and 859-1067 respectively, corresponding to residues 205-281 and 47-116 of cathepsin D. Elevated levels of cathepsin D gene expression were observed in diseased ovarian endometriosis when compared to healthy endometrium for women B and D with Pool C and for women B, D and A with Pool D. High level gene expression of cathepsin D was observed in day 3 (woman D) as well as days 10 and 12 of the cycle (women A and B) indicating that cathepsin D expression might not be hormonally-regulated during the proliferative stage of the cycle in endometriosis. The expression pattern of cathepsin D in these samples was confirmed by an independent method, RT-PCR, using primers specific for the cathepsin D gene (FIG. 3). RT-PCR experiments confirmed the pattern observed for individuals B and D (FIG. 7a). RT-PCR experiments with a larger number of samples proved to be more difficult to interpret due to the abundance of cathepsin D mRNA in both healthy and diseased endometrium that makes a sensitive method such as RT-PCR difficult to discriminate. However, at least three additional patients had elevated cathepsin D mRNA in the diseased tissue. The present data are the most sensitive of all reports, since mRNA levels have been measured and are contradictory to those of Garcia et al. Cathepsin D levels have been found to be elevated in ovarian endometriosis tissue over levels found in healthy tissue, both in the luteal and in the follicular phase.

Elevated levels of this protein are defined herein as being indicative of susceptibility to endometriosis. In order to treat endometriosis, methods should be used that effectively lower cathepsin D activity in diseased tissue. Nucleic acids containing sequence derived from this gene, its complement, the cathepsin D gene product and agonists and antagonists thereof may be used in any of the methods of therapy and diagnosis that are described above.

Accordingly, this aspect of the invention provides for the use of the cathepsin D protein or an active fragment of this protein, for use in the manufacture of a medicament for the treatment or diagnosis of ovarian endometriosis. The invention also provides the use of a nucleic acid molecule containing a cathepsin D gene sequence, nucleic acid molecules that hybridise to this gene sequence and nucleic acid molecules that encode gene products which are functionally equivalent to cathepsin D gene product, in the manufacture of a medicament for the treatment or diagnosis of endometriosis. Such molecules, or agonists and antagonists of these molecules, may also be used in methods of treatment of patients suspected of suffering from endometriosis.

The fact that cathepsin D, an aspartyl proteinase, plays a role in endometriosis indicates that other members of the human aspartic proteinases might be important in the establishment and progression of endometriosis. Such members include pepsinogen A and C, cathepsin E, and renin.

Cystatin B

Cystatin B, an inhibitor of Cathepsin D, has been shown herein to be down-regulated in ovarian endometriosis during days 10 and 12 of the female cycle. This protein is cycle-dependant and appears to be up-regulated during the early proliferative phase of the cycle (patient D). It is hypothesised that an imbalance between cathepsins and cystatins may facilitate cell invasion and metastasis that might be responsible for the establishment of endometriosis, although the Applicant does not wish to be bound by such a theory.

Accordingly, this aspect of the invention provides the use of the cystatin B protein or an active fragment of this protein, for use in the manufacture of a medicament for the treatment or diagnosis of ovarian endometriosis. The invention also provides the use of a nucleic acid molecule containing a cystatin B gene sequence, nucleic acid molecules that hybridise to this gene sequence and nucleic acid molecules that encode gene products which are functionally equivalent to cystatin B gene product, in the manufacture of a medicament for the treatment or diagnosis of endometriosis. Such molecules, or agonists and antagonists of these molecules, may also be used in methods of treatment of patients suspected of suffering from endometriosis.

Protease Inhibitor 1

Alpha-1 antitrypsin, an inhibitor of serine proteases , has been shown herein to be down-regulated in ovarian endometriosis during days 3 and 4 of the female cycle (Table 3). Consistent with the hypothesis presented above, an imbalance between proteases and protease inhibitors may facilitate cell invasion and metastasis of the ectopic endometrium leading to the establishment of endometriosis, although the Applicant does not wish to be bound by such a theory.

Accordingly, this aspect of the invention provides the use of the alpha-1 antitrypsin protein or an active fragment of this protein, for use in the manufacture of a medicament for the treatment or diagnosis of ovarian endometriosis. The invention also provides the use of a nucleic acid molecule containing the alpha-1 antitrypsin gene sequence, nucleic acid molecules that hybridise to this gene sequence and nucleic acid molecules that encode gene products which are functionally equivalent to alpha-1 antitrypsin gene product, in the manufacture of a medicament for the treatment or diagnosis of endometriosis. Such molecules, or agonists and antagonists of these molecules, may also be used in methods of treatment of patients suspected of suffering from endometriosis.

AEBP-1

AEBP-1 cDNA was first identified in human osteoblast and adipose tissue and has been suggested to play a transcriptional repressive role on bone formation. It encodes a protein of 845 amino acids that is almost identical to the mouse adipocyte transcription factor except that it has additional 105 amino acids in the N-terminus.

AEBP-1 (also known as ACLP) has been suggested to be a regulatory B-type carboxypeptidase that effects transcriptional repression. Regulatory carboxypeptidases specifically cleave C-terminal Arg or Lys residues from peptides and proteins and perform a variety of cellular functions including pro-hormone processing, regulation of peptide hormone activity, alteration of protein-protein or protein-cell interactions and transcriptional regulation. It has been suggested that AEBP-1 is a negative transcription factor that regulates transcription by cleaving proteins involved in transcription (He et al., Nature, 1995, 378 (6552): 92-96).

Expression of AEBP-1 has been described on vascular smooth muscle cells of mouse aorta. Vascular smooth muscle cells are the predominant component of the blood vessel wall, with their principal function to regulate vascular tone. AEBP-1 has been implicated in differentiation of vascular smooth muscle cells (Layne et al., J. Biol. Chem., 1998, 273 (25): 15654-15660).

The fact that AEBP-1 has been implicated in vascular smooth muscle cell differentiation together with the present findings that raised levels of AEBP- 1 are observed in at least one benign gynaecological condition might suggest that AEBP-1 plays a role in fibroids. Fibroids is a benign gynaecological condition in which uterine smooth muscle cells proliferate uncontrollably resulting in large benign tumours.

To the best of our knowledge, the presence of AEBP-1 in ovarian endometriotic tissue has never been described. Accordingly, this aspect of the invention provides the AEBP1 protein or an active fragment of this protein, or a gene encoding this protein or an active fragment thereof, for use in the manufacture of a medicament for the treatment or diagnosis of ovarian endometriosis.

Raised levels of AEBP-1 transcript were observed in the diseased ovarian endometriosis tissue of patients A, B and D indicating that gene expression is not hormonally regulated during the proliferative stage of the cycle. The gene fragment identified represents region 657-958 of the AEBP-1 cDNA (dbj: D86479) corresponding to residues 220-320 of AEBP-1.

Elevated levels of this protein are defined herein as being indicative of susceptibility to endometriosis. In order to treat endometriosis, methods should be used that effectively lower AEBP-1 activity in diseased tissue.

Accordingly, this aspect of the invention provides the use of the AEBP-1 protein or an active fragment of this protein, for use in the manufacture of a medicament for the treatment or diagnosis of ovarian endometriosis. The invention also provides the use of a nucleic acid molecule containing an AEBP-1 gene sequence, nucleic acid molecules that hybridise to this gene sequence and nucleic acid molecules that encode gene products which are functionally equivalent to the AEBP-1 gene product, in the manufacture of a medicament for the treatment or diagnosis of endometriosis. Such molecules, or agonists and antagonists of these molecules, may also be used in methods of treatment of patients suspected of suffering from endometriosis.

Stromelysin 3

Stromelysin is a matrix metalloproteinase (MMP-11) that has been implicated in the cyclic breakdown of extracellular matrix that, in women, ultimately leads to menstruation. Although MMPs are tightly controlled by ovarian steroids, MMP-11 has been shown to be induced by several cytokines such as insulin-like growth factor II, epidermal growth factor, platelet derived growth factor and interleukin IL-6 in human endometrial fibroblasts (Singer et al., (1999) Eur. J. Biochem. 259: 40-45).

The identification of stromelysin mRNA in ectopic endometrium (patient D) in this study is particularly interesting. Elevated levels of stromelysin were observed in healthy tissue isolated from women A and B (Day 10 and 12 of the cycle) while stromelysin was absent from ovarian endometriosis tissue isolated from these women. The opposite pattern was observed in woman D (Day 4 of the cycle); no stromelysin mRNA transcribed in healthy tissue but high levels of mRNA on diseased tissue. The RT-PCR experiments confirmed this pattern of expression for patients B and D (FIG. 7b). RT-PCR data was not available for patient A and C due to a shortage of isolated RNA from the diseased tissue of this patient. Subsequent RT-PCR experiments confirmed raised stromelysin levels in healthy endometrium during the proliferative stage and complete depression of stromelysin expression in the secretory phase. Most interestingly, a patient on Zoladex, a GnRH analogue used in treatment of endometriosis, had neither stromelysin in the healthy endometrium nor in the endometriosis.

5 out of the 7 clones sequenced containing the 588 bp differentially expressed fragment identified by Pool D, correspond to stromelysin-3 while the remaining 2 clones represent a fragment of the cystatin B gene, the cathepsin D inhibitor that is discussed above.

The differentially expressed fragment isolated by using Pool D (Table) is the region 240-628 of the human stromelysin-3 mRNA (emb: X57766) corresponding to residues 83-212 of stromelysin-3.

The variation of stromelysin levels in different stages of the cycle is not so surprising considering the hormonal regulation of MMPs during the menstrual cycle. However, the patterns observed in ectopic endometrium in this study have not been described before and indicate misregulated synthesis of MMPs in ectopic endometrium.

There is a study that implicates stromelysin-3 in benign gynaecological conditions in which increased expression levels of stromelysin-3 mRNA are observed in uterine fibroids. mRNA of MMP-11 were determined by semiquantitative RT-PCR in mRNA isolated from uterine fibroids compared with unaffected myometrium (Palmer et al., J Soc Gynecol Investig, 1998, 5 (4) 203- 209). MMP-11 mRNA elevations have been also reported in dermatofibromas compared with unaffected skin. The increased expression of MMP-11 mRNA in fibroid tumours might suggest that stromelysin-3 may be involved in the formation of a more fibrous extracellular matrix in fibroid relative to unaffected myometrium.

Accordingly this aspect of the invention provides the use of the stromelysin-3 protein or an active fragment of this protein for the use in the manufacture of a medicament for the treatment or diagnosis of ovarian endometriosis. The invention also provides the use of nucleic acid molecule containing an stromelysin-3 gene sequence, nucleic acid molecules that hybridise to this gene sequence and nucleic acid molecules that encode gene products which are functionally equivalent to the stromelysin-3 gene product, in the manufacture of a medicament for the treatment or diagnosis of endometriosis. Such molecules, or agonists and antagonists of these molecules, may also be used in methods of treatment of patients suspected of suffering from endometriosis.

Stromelysin-3 levels have been found to be elevated in healthy tissue and absent in endometriosis tissue at day 10 and 12 of the menstrual cycle. However, at least in one patient at the beginning of the cycle (Day 4), these levels are reversed in that no stromelysin-4 mRNA was transcribed in healthy tissue but high levels of mRNA were found in diseased tissue. Accordingly, elevated levels of this protein at the beginning of the menstrual cycle are considered indicative of susceptibility to ovarian endometriosis, whilst depressed levels in the middle of the menstrual cycle are indicative of disease. In order to treat this form of endometriosis, a therapeutic regime should be instigated that lowers stromelysin-3 activity in diseased tissue at the beginning of the cycle and which raises activity towards the middle of the cycle. Alternatively, treatment may be only at the beginning of the cycle. Treatment may include a combination of stromelysin inhibitors with agents that control the cycle, for example, using contraceptive agents.

The fact that at least one member of the matrix metalloproteinases is involved in endometriosis indicates that other members of this family (such as the MMP1-MMP19 proteins) might play a role in the growth, invasiveness, metastasis and angiogenesis of the diseased endometrium.

### Tumour Suppressor Genes

SFRP4

It has also been found that the expression of the gene encoding the Wnt antagonist sFRP4 (secreted frizzled related protein 4) is lowered in ovarian endometriosis tissue.

SFRP4 was not present in diseased ovarian endometriotic tissue isolated from women A and B. Both healthy tissues expressed relatively high levels of SFRP4 mRNA as confirmed by RT-PCR experiments (FIG. 4). Interestingly, the RT-PCR has shown that the reverse trend occurs for samples from patients C and D. SFRP-4 gene expression was minimal or absent in healthy tissues CH, DH and elevated in diseased tissues CR, DV. Considering that patients A and B were at the late proliferative stage of their cycle while patients C and D were at the early proliferative stage of the cycle, SFRP4 expression appears to be hormonally regulated. The SFRP4 fragment found to be differentially expressed in this study correspond to bases 626-871 of the human frpBE mRNA (gb: AF026692) and therefore residues 122-289 of SFRP4.

Additional RT-PCR experiments in a larger number of patients have reinforced the observation that the differential expression of sFRP4 gene is hormonally regulated. Higher levels of sFRP4 were observed in diseased tissue at days 3 and 5 of the cycle consistent with the observation for samples C and D, with the reverse expression pattern observed at days 6 to 24 of the cycle (FIG. 4). sFRP4 levels in only 1 out of 11 patients tested (patient G) was not consistent with this pattern of expression.

This is a particularly interesting finding and appears to be the first observation of the implication of human fizzled-related protein to endometriosis. The data shows that in endometriotic women, FrpHE mRNA is present in Days 6-24 in healthy endometrium and is at lower levels in endometriotic lesions. The absence of this Wnt antagonist from the ovarian endometriotic lesions might indicate the importance of the Wnt-receptor pathway in endometriosis.

Interestingly, a patient treated with Zoladex had elevated sFRP4 mRNA in endometriotic lesions and lower if any sFRP4 mRNA in healthy tissue, indicating the importance of antagonising the Wnt pathway in the treatment of the disease (FIG. 4). Zoladex is a GnRH-analogue that is used in the treatment of endometriosis and acts by reducing estradiol levels.

FrpHE was first identified in endometrial carcinoma by a differential display analysis performed in a collaboration of Beth-Israel-Deaconess Medical Centre and Harvard Medical School (Abu-Jawdeh G et al., Lab. Invest. 1999, 79(4): 439-447). In this study, FrpBE was shown to be hormonally regulated, being present in proliferative endometrium but not significantly detectable in secretory or menstrual endometrium.

This aspect of the invention provides the use of a protein associated with the Wnt-receptor secondary messenger pathway as well as other members of the Wnt (Wnt 1-16) and frizzled (FRP/FrzB) families in the treatment or diagnosis of ovarian endometriosis. Active fragments of such proteins, and the genes encoding these proteins may also be used. This aspect of the invention also provides a protein associated with the Wnt-receptor secondary messenger pathway, active fragments of such proteins, and the genes encoding these proteins or fragments, for use in the manufacture of a medicament for the treatment or diagnosis of ovarian endometriosis.

The following mechanism of action of the Wnt pathway is proposed in endometriosis, although, of course, the Applicant does not wish to be bound by this theory. It is known from studies performed in transgenic mice that wnt-1 has oncogenic potential. Mice transmitting a wnt-1 transgene exhibit extensive hormone-independent hyperplasia of mammary epithelium, suggesting that when wnt-1 binds to its receptor, it induces a number of signalling events resulting in tumour formation. The presence of frizzle protects tissues from tumourigenesis by inhibiting the interaction of wnt with its receptor. In endometriosis, frizzle is down-regulated, failing to inhibit wnt sufficiently in switching on the pathway. Accordingly, a tumour-like phenotype occurs, namely endometriosis.

The sequence of the sFRP4 gene has the accession number: gb: AF026692. Nucleic acids containing sequence derived from this gene, its complement, the gene product and active fragments thereof, and agonists and antagonists thereof may be used in any of the methods of therapy and diagnosis described above.

Depressed levels of the SFRP4 protein are indicative of susceptibility to ovarian endometriosis. In order to treat ovarian endometriosis, methods should be used that effectively raise SFRP4 activity in diseased tissue. Such treatments should take into consideration the hormonal dependency of SFRP4 expression. Alternative methods may be used that inhibit other members of the Wnt pathway in order to inhibit cell growth and other Wnt-related processes in diseased tissue.

The invention also provides the use of a nucleic acid molecule containing an SFRP4 gene sequence, nucleic acid molecules that hybridise to this gene sequence and nucleic acid molecules that encode gene products which are functionally equivalent to the SFRP4 gene product, in the manufacture of a medicament for the treatment or diagnosis of endometriosis. Such molecules, or agonists and antagonists of these molecules, may also be used in methods of treatment of patients suspected of suffering from endometriosis.

Gelsolin

Gelsolin is a Ca²⁺-dependent, lysophosphatidic acid (PIP2) binding protein of approximately 90 kDa that regulates actin filament length by fragmenting actin. Down-regulation of gelsolin has been observed in malignant mammary and prostate tumours and lower levels of gelsolin have been associated with malignant progression. Because of its role in cell motility and growth regulation, gelsolin has been considered as a tumour suppressor molecule.

The present study demonstrates that gelsolin mRNA is upregulated in ovarian and rectovaginal diseased tissues AV and CR while it is absent in healthy tissues AH and CH. There are no reports to date that implicate gelsolin in endometriosis. This is therefore the first report of gelsolin being up-regulated in endometriosis. Considering the function of gelsolin, the discovery that a tumour suppressor is up-regulated in endometriosis may provide one of the reasons that endometriosis is a benign condition.

The involvement of gelsolin in endometriosis suggests that other molecules that are associated with gelsolin may also play a role in endometriosis. Examples include F-actin, PI-3 kinase and c Src (Chellaiah M et al. J. Biol. Chem. (1998) 273(19) 11908-11916).

The invention also provides the use of a nucleic acid molecule containing a gelsolin gene sequence, nucleic acid molecules that hybridise to this gene sequence and nucleic acid molecules that encode gene products which are functionally equivalent to the gelsolin gene product, in the manufacture of a medicament for the treatment or diagnosis of endometriosis. Such molecules, or agonists and antagonists of these molecules, may also be used in methods of treatment of patients suspected of suffering from endometriosis.

### PROTEINS INVOLVED IN CELL GROWTH AND PROLIFERATION

Insulin-like Growth Factor-binding Protein-3 (IGFBP-3)

IGFBP-3 is a member of a family of six well-characterised insulin growth factor (IGF) binding proteins, IGFBP-1 to 6. It is the most abundant IGFBP in the circulation and has been shown to have both IGF-dependent and IGF-independent effects on cell proliferation. IGFBP-3 modulates the interaction of IGFs and their cell surface receptors, resulting in either inhibition or stimulation of cellular growth. IGFBP-3 has also been shown to inhibit cell growth in the absence of IGFs (Fanayan et al., 2000, JBC September 18 [e-pub ahead of print]).

IGFBP-3 has been recently implicated in inducing apoptosis in breast cancer cells (Butt et al. (2000) (JBC September 20 [e-pub ahead of print]). When Butt et al. demonstrated that IGFBP-3 upregulates the expression of the pro-apoptotic Bax protein and downregulates the anti-apoptitic proteins Bcl-2 and Bcl-xL.

The present study demonstrates that IGFBP-3 mRNA is downregulated in ovarian and rectovaginal diseased tissues DV and CR and it is absent in both healthy and diseased tissues of patients A and B (Days 10 and 12). The absence of IGFBP-3 mRNA in patients A and B is not surprising as the inhibitory effect of estradiol on IGFBP-3 protein and mRNA levels has been reported by several workers (Liu et al., Mol. Hum. Reprod. (1997) 3 No.1, 21-26; Huynh & Pollak, Cancer Res. (1994) 54 (12), 3115-3119). Liu et al. demonstrated the inhibitory effect of estradiol on protein and mRNA levels of IGFBP-3 on secretory endometrial stromal cell cultures treated with various hormones while Huynh & Pollak demonstrated that estradiol and tamoxifen supress IGFBP-3 gene expression in uterus to less than one third of control values, while oophorectomy or administration of estrogen receptor antagonist result in greater than 3 fold stimulation of uterine IGFBP-3 gene expression. Additionally, IGFBP-3 mRNA has been shown to be primarily concentrated in the endometrial capillaries and to be increased in the secretory phase, largely due to the intense vascularisation of endometrial glands during this phase (Zhou et al., J. Clin. Endocrinol.Metab., (1994) 79, No.6, 1723-1734).

A number of studies has focused on determining the levels of IGFBP-3 protein in the serum and peritoneal fluid of patients with endometriosis by immunoradiometric assays and Western blots. However, the interpretation of these data is complicated by the fact that IGFBP-3 levels are determined not only by mRNA expression but also by posttranslational proteolytic degradation. Proteolytic degradation of IGFBP-3 was first shown in the peritoneal fluid and serum from normally cycling women when Western immunoblotting revealed IGFBP-3 forms of 37-43 kDa (major) and 28 kDa (minor) in serum and almost exclusively the 28 kDa band in peritoneal fluid (Giudice et al., J. Clin. Endocrinol. Metab. (1994) 79 (5) 1284-1293). When the same methods were applied in patients with endometriosis and compared to control groups, the data were contradictory, with some groups suggesting that the levels of IGFBP-3 are significantly lower in patients with endometriosis (Kim et al., Fertil Steril May 2000, 73 (5) 996-1000), and other groups suggesting no differences between endometriosis and control patients. (Gurgan et al., J. Reprod. Med. May 1999 44(5) 450-454).

To the best of our knowledge, this is the first report suggesting that IGFBP-3 mRNA is downregulated in endometriosis. Downregulation of IGFBP-3 expression in endometriosis may be responsible for an increased mitogenic effect of IGF in endometriosis although the inventors do not wish to be bound by this theory. The estradiol dependence of IGFBP-3 expression seen in the eutopic endometrium is suggestive that the ectopic endometrium is not hormonally regulated in a similar manner to the eutopic endometrium. In order to treat endometriosis, methods should thus be used that effectively increase IGFBP-3 protein activity, preferably in diseased tissue. Such methods might include effecting raised levels of IGFBP-3, or of IGFBP-3 protease inhibitors. The downregulation of IGFBP-3 observed in ectopic endometrium in this study, implicates IGF playing a role in endometriosis. An alternative method for treating endometriosis may therefore be to antagonise IGF directly.

The invention also provides the use of a nucleic acid molecule containing an IGFBP-3 gene sequence, nucleic acid molecules that hybridise to this gene sequence and nucleic acid molecules that encode gene products which are functionally equivalent to the IGFBP-3 gene product, in the manufacture of a medicament for the treatment or diagnosis of endometriosis. Such molecules, or agonists and antagonists of these molecules, may also be used in methods of treatment of patients suspected of suffering from endometriosis.

Dual Specificity Phosphatase 1

Dual specificity phosphatase 1 plays a role in regulating mitogen-activated protein kinase (MAPK) activity by dephosphorylating phosphorylated threonine and tyrosine residues of the kinase. MAP kinases relay both proliferative, via extracellular regulated kinases (ERK), as well as apoptotic signals, via jun N-terminal protein kinases (JNK), to the nucleus. Dual specificity phosphatase 1 may play therefore an antiproliferative or an anti-apoptotic role depending on which MAP kinase it dephosphorylates. As an example, it has been proposed that dual specificity phosphatase 1 inhibits apoptosis in human prostate tumours, possibly through the JNK pathway (Magi-Galluzi C et al., Lab. Invest. 1997, Jan; 76(1): 37-51).

Remarkably, increased levels of dual specificity phosphatase 1 have been observed in diseased tissue of five out of six patient samples. Endometriotic lesions of patients D (Day 3), N (Day 5), K (Day 6), B (Day 12) and P (Day 15) all had higher levels of dual specificity phosphatase 1 mRNA in comparison to eutopic endometrium suggesting that dual specificity phosphatase 1 is elevated in endometriosis during the entire proliferative phase of the cycle. The differentially expressed fragment consists of bases 669-1155, corresponding to residues 140-301 of the protein.

To the best of our knowledge, this is the first report suggesting dual specificity phosphatase 1 mRNA is upregulated in endometriosis. Upregulation of dual specificity phosphatase 1 expression in endometriosis may implicate the involvement of the MAP kinase pathways in endometriosis although the inventors do not wish to be bound by this theory.

Elevated levels of this protein are defined herein as being indicative of susceptibility to endometriosis. In order to treat endometriosis, methods should thus be used that effectively decrease dual specificity phosphatase 1 activity, preferably in diseased tissue. Such methods might include the use of an inhibitor in the manufacture of a medicament for the treatment of endometriosis. Alternatively methods increasing MAP kinase activity in the endometriotic lesions may be used to effectively decrease the effect of the overexpression of dual specificity phosphatase 1 in the endometriotic lesions.

The invention also provides the use of a nucleic acid molecule containing a dual specificity phosphatase 1 gene sequence, nucleic acid molecules that hybridise to this gene sequence and nucleic acid molecules that encode gene products which are functionally equivalent to the dual specificity phosphatase 1 gene product, in the manufacture of a medicament for the treatment or diagnosis of endometriosis.

### Proteins with a Function in the Immune System or in Inflammatory Responses

Immunoglobulin Lambda

Immunoglobulins contain heavy and light chains (H and L). All chains contain variable and constant regions. Various splicing events take place to generate the variable sequences that are responsible for the extensive diversity which enables the immune system to recognise and react with foreign antigens. There are two families of light chains referred to as kappa and lambda chains (see Fundamental Immunology (1999), W. E. Paul, Lippincott-Raven, Philadelphia; New York). While the DNA encoding the portions of the lambda chains is used predominantly in the formation of light chains, some sequences have been shown to involved in complex switching patterns that also involve the heavy chains. The term surrogate light chains has been used. Light chains are known to be involved in the translocation of immunoglobulins to the surface of B-cells where they play an important part in the recognition of antigens. Lambda light chains are not synthesised until later in an immune reaction and it has been postulated that surrogate light chains become associated with heavy chains in order to achieve the translocation of antibody to the surface of B-cells in advance of the complete synthesis of light chains (see Sakaguchi, N & Melcher, F. (1986) Nature, 372, 579-582).

In the present study, elevated expression of the immunoglobulin-λ gene was observed in diseased endometriotic tissue from women A, B and D indicating that the raised levels of immunoglobulin lambda gene were independent of hormonal levels during the early as well as late proliferative stage of the cycle. These results were confirmed with RT-PCR using primers (see FIG. 3) specific for the immunoglobulin-λ chain sequence (Y14737). (FIG. 5). There was not any immunoglobulin-λ light chain transcript found in any of the healthy samples A, B and D as shown by the RT-PCR (FIG. 5).

Subsequent RT-PCR experiments in a larger number of patients confirmed elevated expression of the immunoglobulin λ gene in 4 out of 7 patients at the early and late proliferative stage of the cycle (Day 3-15). Interestingly, the two patients G and I that showed the reverse expression profile (elevated expression in healthy rather than diseased) were both at the secretory phase of the cycle Days 16 and 24 respectively (FIG. 5).

Differential expression was observed in diseased tissue irrespectively of whether the tissue was of rectovaginal or ovarian disease state. Elevated levels of this protein are thus indicative of susceptibility to endometriosis.

In order to treat endometriosis, methods should be used that effectively lower immunoglobulin λ light chain, preferably in diseased tissue. Such methods might include the use of immunosuppressants.

The invention also provides the use of a nucleic acid molecule containing an immunoglobulin λ light chain gene sequence, and nucleic acid molecules that hybridise to this gene sequence.

Complement Component-3

Complement component-3 synthesis and secretion from endometrium of patients with and without endometriosis has been described (Sayegh R A et al., J. Clin. Endocrinol Metab, 1996, 81(4) pg 1641-1649; Isaacson et al., 1990, Fertil Steril 53(5) pg836-8⁴1; Issacson et al., J. Clin. Endocrinol. Metab. 1989, 69(5) pg 1003-1009).

More recently, mRNA of complement component 3 in human eutopic and ectopic endometrium has been compared by in situ hybridisation experiments and found to be significantly increased in endometriotic lesions compared with that in the eutopic endometrium (Tao X J et al., Fertil Steril 1997 68(3) pg 460-467).

In this study, raised levels of complement component 3 were found in the diseased endometrium of five out of six patients, D, N, B, P and I by DDRT-PCR. The differentially expressed fragment of complement component 3 was containing bases 4328-4770 corresponding to residues 1422-1569. Patients D, N, B, P and I span the entire cycle, from early proliferative to middle secretory suggesting that complement component 3 gene expression is not hormonally regulated in the endometriotic lesions.

The agreement of the DDRT-PCR data obtained herein with the data of Tao et al. provide confirmatory evidence of the link between complement component 3 and endometriosis.

Elevated levels of this gene are defined herein as being indicative of susceptibility to endometriosis. The invention provides the use of a nucleic acid molecule containing a complement componet 3 gene sequence, nucleic acid molecules that hybridise to this gene sequence and nucleic acid molecules that encode gene products which are functionally equivalent to the complement component 3 gene product, in the manufacture of a medicament for the treatment or diagnosis of endometriosis.

Ferritin

One example of a protein implicated in inflammation that is herein connected to ovarian endometriosis is ferritin. Ferritin is an iron-binding protein that plays a major role in cellular homeostasis. It is composed of two subunits, heavy (H) and light (L) chain. Ferritin has been implicated in acute inflammation and there are several studies reporting on elevated serum ferritin levels in patients with inflammation and chronic disease (Tran et al. Blood (1997) 90 (12) pg 4979-4986). The induction of the ferritin light chain encoding gene in an inflammatory disease (rheumatoid arthritis) was first reported by Heller et al. (PNAS, (1997) 94, pg 2150-2155).

The data provided herein demonstrate up-regulation of both ferritin L and H-chain genes in endometriosis. Although only one out of 10 clones represented the ferritin L-chain gene sequence, RT-PCR experiments using primers specific for the ferritin gene sequence were consistent with elevated levels of ferritin L-chain in women B and D. (FIG. 7a) Subsequent RT-PCR experiments in a larger number of patients confirmed elevated ferritin mRNA levels in 4 out of 6 diseased tissues tested. The Indexing DDRT-PCR using a different adapter pool was consistent with the ferritin H-chain also being up-regulated in diseased tissue from women A, B and C. Endometriotic samples with raised levels of ferritin were from the proliferative to the mid-secretory stage of the cycle, suggesting that ferritin gene expression is not hormonally regulated; however, the inventors do not wish to be bound to this theory.

Elevated levels of this protein may suggest that ferritin receptors are raised in the endometriotic lesions. In this case, ferritin may be used in order to deliver toxic compounds to endometriotic lesions as a method of therapy for endometriosis.

Elevated levels of ferritin H and/or L chains are defined herein as being indicative of susceptibility to endometriosis. Determination of ferritin L and/or H chain gene product as well as serum iron levels should provide a particularly useful diagnostic tool. In order to treat endometriosis, methods should thus be used that effectively decrease ferritin levels, preferably in diseased tissue.

The invention also provides the use of a nucleic acid molecule containing ferritin H and/or L chain gene sequence and nucleic acid molecules that hybridise to this gene sequence in diagnosis of endometriosis.

Human Progestagen-dependent Pregnancy-associated Endometrial Protein

A further example of the involvement of a protein with a potential function in the immune system in endometriosis may be found in the human progestagen-dependent pregnancy-associated endometrial protein (PAEP or PP14 or glycodylin) that has been found to be altered in endometrium tissue from diseased patients.

This aspect of the invention provides the PAEP protein or an active fragment of this protein, or a gene encoding this protein or active fragment, for use in the manufacture of a medicament for the treatment or diagnosis of endometriosis. Nucleic acids containing sequence derived from this gene, its complement, the gene product and active fragments thereof, and agonists and antagonists thereof may be used in any of the methods of the invention described above.

PAEP is a hormone dependent endometrial protein observed in reproductive-associated tissues. In healthy individuals, PAEP levels rise from the early to the late luteal phase (˜Days 14-28), being highest at the time of menstrual bleeding. Elevated levels of PAEP have been also observed at the first trimester of pregnancy and it has been suggested that PAEP plays an immunosuppressive role in human reproduction.

PAEP mRNA was first identified in ectopic endometrium by in situ hybridisation (J. Mol. Endocrinol., 1993, 10 (1) pg 71-77; Hum. Reprod., 1990, 5 (5) pg 487-493). The data provided herein contradict the results of this study since no PAEP mRNA was observed in diseased tissue, whilst PAEP mRNA was seen in healthy tissue. In the above study, the two endometriosis samples tested were from a 21 weeks pregnant woman and from a borderline endometrioid adenoma. These biopsies may therefore not represent classic endometriotic lesions.

Telimmaa et al. observed elevated levels of PAEP at the early stage of the cycle (Am J Obstet Gynecol, 1989, 161 (4) 866-871). These authors measured serum levels of PAEP in patients with advanced endometriosis and observed highest levels of PAEP on days 1-4 of the cycle and lowest levels on days 5 to 20, speculating that the endometriotic lesions actually contribute to the serum PAEP levels.

The data provided herein demonstrate that PAEP mRNA is downregulated in ovarian and rectovaginal lesions of patients C and D. This was shown twice with two independent adaptor pools (Table 3). The RT-PCR results were consistent with the observation that healthy tissue from individuals C and D were expressing PAEP while the diseased tissue did not express PAEP (FIG. 5b). Interestingly, RT-PCR experiments have also shown that higher levels of PAEP mRNA were produced from the healthy tissues isolated from individuals A and B. Subsequent RT-PCR experiments in a larger number of patients confirmed the presence of PAEP in healthy endometrium and its absence from diseased endometrium in 7 out of 7 patients at days 3-24 of the cycle (FIG. 5b). Interestingly, treatment with Zoladex eliminated the transcription of any PAEP in healthy tissues.

To the best of our knowledge this is the first report demonstrating complete absence of PAEP mRNA in endometriotic lesions.

The double band observed in the RT-PCR experiments performed herein indicate the presence of spliced forms of PAEP (FIG. 5b). Spliced variants for PAEP were reported although not characterised by Garde et al. (PNAS 88, 2456-2460, 1991). In the presence of these data, it is thought likely that the elevated levels of PAEP found by Telimmaa et al. in early proliferative stage are in fact carried over from the high levels present at the end of the luteal phase of the cycle.

In order to treat endometriosis according to this aspect of the invention, methods should be used that effectively raise PAEP activity in diseased tissue. Nucleic acids containing sequence derived from this gene, its complement, the gene product and active fragments thereof, and agonists and antagonists thereof may be used in any of the methods of therapy and diagnosis that are described above.

### TRANSCRIPTION/TRANSLATION REGULATORY FACTORS

Human Elongation Factor-1 (EF-1 alpha)

A specific example of a transcription/translation regulatory factor implicated in endometriosis is elongation factor-1, alpha subunit.

Human elongation factors are an important house-keeping enzyme which have been assigned a number of important biological functions of the cell. Elongation factors are involved in protein synthesis by mediating the transport of aminoacyl tRNA to 80S ribosomes and therefore high levels of an elongation factor may be suggestive of high transcirptional/translational activity in a tissue. Overexpression of polypeptide chain initiation factors as well as the involvement of elongation factor eEF1 in oncogenesis have been recently reviewed (Clemens et al., (1999) Int J Biochem Cell Biol, 31(1): 1-23).

High expression levels of elongation factor has been reported in a large number of tumours, but most interestingly, higher levels of elongation factor 1 have been observed in benign rather than malignant breast tumours (Adams et al., (1992) Cancer 65(1): 65-71).

This aspect of the invention provides the elongation factor 1 alpha subunit protein or active fragments of this protein, or a gene encoding this protein or on active fragment thereof, for use in the manufacture of a medicament for the treatment or diagnosis of endometriosis.

Levels of elongation factor-alpha subunit gene expression have been found to be raised in healthy tissue of patients A, B and D indicating that gene expression of elongation factor is raised at both the early as well as late proliferative stage of the cycle (FIG. 7e). The fragment identified corresponds to bases 389-680 of the human elongation factor-alpha subunit gene (emb:X03558) and therefore residues 111-226 of human elongation factor. A second fragment of elongation factor, alpha subunit was identified from a different adapter pool corresponding to bases 764-1097 of the human elongation factor alpha subunit gene. This fragment was differentially expressed in the same manner as the first one confirming that levels of elongation factor gene expression were raised in healthy tissues of patients A, B and D. Subsequent RT-PCR experiments proved to be difficult to show consistent differences in a large number of patients most likely due to the abundance of the elongation factor gene in both eutopic and ectopic endometrium that make a sensitive method such as RT-PCR difficult to discriminate.

Accordingly, elevated levels in the eutopic endometrium of this protein are considered to be indicative of susceptibility to ovarian endometriosis. In order to treat ovarian endometriosis, methods should be used that effectively lower elongation factor activity in healthy tissue. Nucleic acids containing sequence derived from this gene, its complement, the gene product and agonists and antagonists thereof may be used in any of the methods of therapy and diagnosis that are described above.

Nascent-polypeptide-associated Complex Alpha Polypeptide (NACA)

Nascent polypeptide associated complex (NAC) is a cytosolic protein that is involved in protein translation, binding to polypeptides as they emerge from ribosomes. NAC consists of two copurifying polypeptides termed as alpha (NACA) and beta that form a very stable complex. Although NACA does not bind to ribosomes, it has been suggested that it interacts with nucleic acids including those present in ribosomes, although its affinity is not restricted to DNA and is not sequence specific (Beatrix et al., 2000, Sep. 1 1 Fe-pub prior to print]). Interestingly the NACA gene has been recently implicated in malignant brain tumours (Kroes et al., Cancer Lett 2000, 156(2) 191-198). These authors discovered differential expression of NACA mRNA in normal brain in comparison to primary tumour tissues.

The present study is the first report of NACA playing a role in endometriosis. Levels of NACA gene expression have been found to be raised in diseased ovarian and rectovaginal endometrial tissue of patients A, B and C indicating that gene expression of NACA is raised at both the early as well as late proliferative stages of the cycle.

Accordingly, elevated levels of this protein are indicative of susceptibility to endometriosis. In order to treat endometriosis, methods should be used that effectively lower NACA activity in diseased tissue. Nucleic acids containing sequence derived from this gene, its complement, the gene product and agonists and antagonists thereof may be used in any of the methods of therapy and diagnosis that are described above.

### ENZYMES

In a further embodiment of the invention, it has been discovered that various genes encoding enzymes have a role in ovarian endometriosis. One example is vitamin D3 25 hydroxylase. This aspect of the invention therefore provides vitamin D3 25 hydroxylase or active fragments of this enzyme, or a gene encoding this protein or active fragments thereof, for use in the manufacture of a medicament for the treatment or diagnosis of ovarian endometriosis.

Vitamin D3 25 Hydroxylase

Vitamin D3 25 hydroxylase is a 500 amino acid protein responsible for the first step in the metabolic activation of vitamin D3 into its hormonal form 1α25(OH)D3. Although vitamin D metabolites have been extensively studied in endometriotic women, investigations to date have shown that expression of vitamin D3 hydroxylase from ectopic endometriotic tissue has not been reported.

In one study, 18 patients with endometriosis and uterine fibroids received GnRH-agonists and the levels of 25- and 1,25-dihydroxyvitamin D3 were monitored. Levels of 1,25-dihydroxyvitamin D3 decreased significantly, but 25-hydroxyvitamin D3 values remained constant (Waibel-Treber et al., Hum. Reprod., 1989, 4 (4): 384-388). It is not clear whether or not these values are due to over-expression of vitamin D3 25 hydroxylase from endometriotic tissue observed in our study.

Expression of this gene has been found to be elevated in diseased endometriosis tissue from patients B and D suggesting that vitamin D3 25-hydroxylase expression is not hormonally regulated during the proliferative stage of the cycle (Table 3,FIG. 7d). The differentially expressed fragment corresponds to base pairs 379-656 of the vitamin D3 25-hydroxylase mRNA (emb: X59812) and consequently residues 59-151 of vitamin D3 25-hydroxylase.

Accordingly, elevated levels of this protein or/and its metabolites are indicative of susceptibility to ovarian endometriosis. In order to treat ovarian endometriosis, methods should be used that effectively lower vitamin D3 25 hydroxylase activity in diseased tissue. Nucleic acids containing sequence derived from this gene, its complement, the gene product and agonists and antagonists thereof may be used in any of the methods of therapy and diagnosis that are described above.

### LIPID BINDING PROTEINS

In a further embodiment of the invention, it has been discovered that various genes encoding lipid binding proteins have a role in ovarian endometriosis. Examples include steroidogenic acute regulatory protein, apolipoprotein E, and prosaposin. This aspect of the invention therefore provides steroidogenic acute regulatory protein, apolipoprotein E and prosaposin or active fragments thereof, or a gene encoding these proteins or active fragments, for use in the manufacture of a medicament for the treatment or diagnosis of ovarian endometriosis.

Steroidogenic Acute Regulatory Protein (StAR)

Steroidogenic acute regulatory protein (StAR) plays a key role in steroid hormone synthesis by enhancing the metabolism of cholesterol into pregnenolone by mediating translocation of cholesterol from the outer to the inner mitochondrial membranes. Pregnenolone is metabolised to progesterone or/and 17a-OH-pregnenolone, the precursor molecule for the synthesis of estradiol and testosterone.

Recurrence of endometriosis quickly after cessation of medical therapy as well as the persistence of histologically active lesion even after six months of GnRH agonist therapy has been suggestive of endometriotic lesions being hormone-independent. Attempts have been made to explain the hormonal independence of ectopic endometrium by an abnormal steroid receptor regulation, as suggested by the persistence of a high glandular progesterone receptor content during the late secretory cycle. To the best of our knowledge, this is the first study implicating steroidogenic acute regulatory protein, a key enzyme in steroidogenesis, in endometriosis.

Levels of steroidogenic acute regulatory protein gene expression have been found to be raised in five out of six diseased endometrial tissue of patients B, D, N, P and I indicating that gene expression of steroidogenic acute regulatory protein is consistently raised during the proliferative and mid-secretory stage of the cycle. The differentially expressed fragment identified by DDRT-PCR corresponds to 3′-end of the steroidogenic acute regulatory protein gene (NM_{—}000349). All healthy tissues of patients B,D,N, P and I did not contain detectable amount of steroidogenic acute regulatory protein by DDRT-PCR.

In order to treat endometriosis according to this aspect of the invention, methods should be used that effectively depress or/and inhibit steroidogenic acute regulatory protein activity in diseased tissue. Nucleic acids containing sequence derived from this gene, its complement, the gene product and active fragments thereof, and agonists and antagonists thereof may be used in any of the methods of therapy and diagnosis that are described above.

Apolipoprotein E

Apolipoprotein E (apoE) is a 34-kDa polypeptide that is involved in cholesterol metabolism. It has been suggested to protect against atherosclerosis, in part because apoE phospholipid particles secreted by macrophages may have local protective effects within lesions. Although apoE is an extensively studied protein, there are not thought to be any studies implicating apoE in ovarian endometriosis.

Expression of this gene has been found to be elevated in diseased ovarian endometrial tissue from patients B and D. Considering that apoE mRNA levels were raised at both the early as well as late proliferative stage suggest that apoE expression is not oestrogen regulated. The differentially expressed gene fragment sequence corresponds to base pairs 3-141 of the human apoE mRNA (emb: X00199) and consequently residues 2-46 of apoE.

Accordingly, elevated levels of this protein are indicative of susceptibility to ovarian endometriosis. In order to treat ovarian endometriosis, methods should be used that effectively lower apolipoprotein E activity in diseased tissue. Nucleic acids containing sequence derived from this gene, its complement, the gene product and agonists and antagonists thereof may be used in any of the methods of therapy and diagnosis that are described above.

Prosaposin (SAP1)

Prosaposin is a 65 kDa glycoprotein that contains separate domains in tandem for four lysosomal saposins A, B, C and D. These saposins are sphingolipid activator proteins (SAPs) derived from their precursor saposin by proteolytic processing. These small (12-14 kDa) glycoproteins are required for lysosomal hydrolysis of a variety of glycosphingolipids by exohydrolases. They are thought to function either by direct activation of their respective enzymes or as biological detergents that solubilize the lipids out of the membrane.

High expression of the prosaposin gene has been observed in adult and embryonic nervous and reproductive systems and has been found to be expressed in the mature female gonads at various stages of the corpus luteum development.

Expression of the prosaposin gene has been found to be elevated in diseased ovarian endometrial tissue from patients A, B and D. The differentially expressed gene fragment sequence corresponds to base pairs 1545-1761, corresponding to the last twelve C-terminal amino acids of prosaposin and a sequence of the 3′ untranslated region of the human prosaposin mRNA (J03015).

Considering that saposin A, B, C and D are the proteolytically cleaved products of prosaposin, we can only assume from these data that the overexpression of prosaposin in the ectopic endometrium results in overproduction of all four saposins in endometriosis. However, a further glycosylated form of prosaposin (70 kDa) gets secreted and is not targeted to the lysosomes. Secreted prosaposin has been found in breast cancer cells (Campana et al., Biochim. Biophys. Acta (1999) Vol. 1427, No.3, 392-400) and it has been speculated that together with other lysosomal proteins in the tumour environment, such as procathepsin D, this protein may act as a factor in eliminating barriers to tumour metastasis by facilitating hydrolysis of membrane glycolipids. Considering that endometriosis resembles metastasis, in that eutopic endometrium migrates and implants outside the uterine cavity, the elevated prosaposin seen in endometriotic lesions might facilitate their implantation and maintenance by locally solubilising glycolipids of the peritoneal or ovarian surface.

Accordingly, elevated levels of this protein are considered indicative of susceptibility to ovarian endometriosis. In order to treat ovarian endometriosis, methods should be used that effectively lower prosaposin activity in diseased tissue. This may be achieved by administering glycosphingolipid mimic molecules that will bind with high affinity to prosaposin and inhibit this protein from solubilising glycolipids of the peritoneal or ovarian surface, subsequently prohibiting lesion implantation. Nucleic acids containing sequence derived from this gene, its complement, the gene product and agonists and antagonists thereof may be used in any of the methods of therapy and diagnosis that are described above.

### MATRIX AND CELL ADHESION PROTEINS

In a further embodiment of the invention, it has been discovered that genes encoding matrix and cell adhesion proteins have a role in endometriosis. For example, expression of the gene encoding the matrix protein, pro-alpha-1 type III collagen and proline 4-hydroxylase beta polypeptide have been found to be lowered in diseased endometrium tissue, both from ovarian and recto-vaginal tissue. Collagen alpha-2 type I has been found to be downregulated in diseased endometrium tissue in certain days of the cycle and the reverse expression pattern was observed near and just after menstruation. Examples of genes involved in cell adhesion are the genes encoding for claudin-4, a transmembrane protein directly involved in cell adhesion and the formation of tight junctions and melanoma adhesion protein, a transmembrane glycoprotein involved in cohesion of the endothelial monolayer. Claudin-4 has been found to be downregulated in endometriosis when compared to eutopic endometrium while melanoma adhesion protein has been found to be upregulated in the endometriotic lesions.

### Collagen Alpha-1 Type III

Collagen is an extracellular protein that is organised into insoluble fibres, which act as the major stress-bearing component of connective tissues such as bone, tendon and cartilage. Several heritable disorders of collagen are known to result from mutations of type-I collagen or deficiencies in the amount of a particular collagen type synthesised. Many degenerative diseases exhibit collagen abnormalities in the affected tissues (for example, osteoarthritis and atherosclerotic plaques).

The involvement of collagen in endometriosis has been previously described with both type-I and type-Ill collagen found in the stroma of peritoneal endometriotic implants, with only type-I collagen found in the collagenous tissue surrounding endometriotic implants (Matsuzaki et al.; Gynecol. Obstet, Invent., 1999, 47(3) 197-199). In this study, type-III collagen was present in the healthy endometrium and absent from either the ovarian or the rectovaginal ectopic endometrium. This observation was verified by using three independent pools (Table 3). All three pools confirmed the downregulation of collagen type III alpha 1 mRNA in diseased tissue from patients A, B and C (see Table 3 and FIG. 7c). Two of these pools, pool E and pool J, identified the same fragment (2858-3336 corresponding to residues 918-1078) being absent in endometriotic lesions while the third pool identified fragment 1936-2218, corresponding to residues 611-715 Subsequent, RT-PCR experiments confirmed reduced levels of type 3 collagen in ectopic endometrium of 4 out of 6 patients.

The first study describing the involvement of type-Ill collagen in ovarian endometriosis was performed by Khare et al. (J Am. Assoc. Gynecol. Laparosc., 1996, 3 (2) 235-239). The cases of ovarian and pelvic wall infiltrating endometriosis were studied by histological examination and the presence of loose myxoid type-III collagen was identified in ovarian sections but not on the pelvic wall infiltrating endometriosis. In contrast to these results, the present study observed the absence of collagen type-III from ovarian endometriosis. This may be due to the fact that an earlier stage of endometriosis was studied than the stage studied by Khare et al., that focuses on deep infiltrating endometriosis.

This aspect of the invention therefore provides the pro-alpha-1 type III collagen protein or an active fragment of this protein, or a gene encoding this protein or an active fragment thereof, for use in the manufacture of a medicament for the treatment or diagnosis of endometriosis.

Depressed levels of this protein are therefore defined herein as being indicative of susceptibility to endometriosis. In order to treat endometriosis, methods should be used that effectively raise pro-alpha-1 type III collagen activity in diseased tissue. Nucleic acids containing sequence derived from this gene, its complement, the gene product and active fragments thereof, and agonists and antagonists thereof may be used in any of the methods of therapy and diagnosis that are described above.

Collagen Alpha-2 Type I and Procollagen C-endopeptidase Enhancer

Type I collagen is the most abundant proteins in the mammalian body. It consists of three subunits, two identical alpha-2 subunits, encoded by gene COL1A2, and one alpha-1 subunit encoded by gene COL1A1. Mutations in the COL1A1 and COL2A2 genes cause a range of diseases including mild to lethal forms of osteogenesis imperfecta and a set of Ehlers-Danlos syndrome. Although the presence of type I collagen has been described in endometrium (Iwahashi M et al., 1996, 108(1):147-155), alpha-2 type I collagen has never been implicated in endometriosis before.

Alpha-2 type I collagen mRNA levels were found to be downregulated in diseased tissues from patients K and B (Days 6 and 12). The opposite pattern of expression was observed for patients D and I (Days 3 and 24 respectively). This differential expression at different stages of the cycle might be indicative of hormonal regulation of alpha-2 type I collagen expression. Most importantly, the differential expression observed in endometriotic lesions suggest that extracellular matrix development in the endometriotic lesion is different than in the endometrium, although the inventors do not wish to be bound by this theory.

Type I procollagen C-terminal proteinase enhancer, a glycoprotein that binds to the carboxy-terminal propeptide of type I procollagen, appeared to have a similar pattern of expression to that observed for the type I alpha-2 collagen. Downregulation of protease expression was observed in the ectopic endometriotic lessions in four out of five endometriosis patients (Table 2). Interestingly both the human proteinase enhancer gene, PCOLCE, as well as the type I alpha-2 collagen chain gene, COL1A2 are localised to the same chromosomal region 7q21.3→q22 (Takahara et al., 1994, JBC 269 (42) pg 26280-26285). A very recent study has suggested a possible role of the PCOLCE gene in fibroids, as leiomyomas are frequently disrupted at the 7q22 region.

Depressed levels of this protein are therefore defined herein as being indicative of susceptibility to ovarian and recto-vaginal endometriosis. In order to treat these forms of endometriosis, methods should be used that effectively raise alpha-2 type I collagen or procollagen C-terminal proteinase enhancer activity in diseased tissue. Nucleic acids containing sequence derived from this gene, its complement, the gene product and active fragments thereof, and agonists thereof may be used in any of the methods of therapy and diagnosis that are described above.

Proline 4-hydroxylase Betapolypeptide

Proline 4-hydroxylase beta polypeptide gene expression has been found in this study to be downregulated in endometriotic lesions in four patients (A, B, C and D) (Table 3). Prolyl 4-hydroxylase is a tetramer consisting of alpha-2 and beta-2 subunits that catalyses the formation of 4-hydroxyproline in collagens by the hydroxylation of proline residues in peptide linkages; 15-30% of collagen residues are proline and 4-hydroxyproline. Proline 4-hydroxylase plays a crucial role in conferring stability upon collagen by hydroxylating the proline residues of collagen post-translationally. Hydroxyproline is essential for maintaining collagen conformation at 37° C.; collagen synthesised under conditions that inactivate prolyl 4-hydroxylase denatures at 24° C.

The downregulation of proline 4-hydroxylase betapolypeptide observed here in endometriotic lesions suggests that those lesions have a deficiency in the formation of stable collagen structures. This taken together with the observation that collagen type I and type III are downregulated in endometriosis suggest that the endometriotic lesions are defective in forming stable extracellular matrix. The inventors herein suggest that the defects of the extracellular matrix of endometriotic lesions reported here may play an important role in the implantation or invasion of the endometriotic lesion, however, they do not wish to be bound to this theory.

Although, proline-4 hydroxylase has been reported to be located in the cytoplasm of endometrial stromal cells and endometrial glandular cells during the menstrual cycle in endometrium (Iwahashi M et al., J. Reprod. Fertil. 1996 September;108(1) pg 147-155), there are no reports implicating proline-4 hydroxylase deficiency in endometriosis.

Depressed levels of this protein are therefore defined herein as being indicative of susceptibility to ovarian endometriosis. In order to treat these forms of endometriosis, methods should be used that effectively raise proline 4-hydroxylase activity in diseased tissue. Nucleic acids containing sequence derived from this gene, its complement, the gene product and active fragments thereof, and agonists thereof may be used in any of the methods of therapy and diagnosis that are described above.

Melanoma Adhesion Molecule (MCAM, CD146)

MCAM is a transmembrane glycoprotein expressed in endothelial cells and located in the intercellular junction functions as a calcium independent cell adhesion molecule. MCAM has been shown to play an important role in melanoma tumour progression, influencing later stages of the metastatic process by increasing cell adhesion leading to formation of tumour cell clusters. Recently, high levels of soluble MCAM have been found in rheumatoid arthritis synovial fluid in patients with early disease and it has been suggested that they reflect increased activity of endothelial cells and angiogenesis (Neidhart et al., 1999, Arthritis Rheum. 42(4) pg 622-630).

MCAM mRNA levels were found to be higher in diseased tissue from patients K, P and I than in healthy tissue KH, PH and IH. The cloned fragment identified as MCAM represents bases 1919-2315 at the 3′-end of the human mRNA for MCAM (NM_{—}006500).

Increased levels of MCAM in endometriosis are suggestive of formation of endothelial intracellular junctions and potentially increased vasculogenesis. MCAM is expressed on endothelial cells on the whole vascular tree. Increased cohesion of the endothelial cell monolayer may allow lesion progression. To the best of our knowledge, the present study is the first report implicating MCAM in endometriosis.

Increased levels of this protein are therefore defined herein as being indicative of susceptibility to endometriosis. In order to treat or relieve the symptoms of endometriosis, methods should be used that effectively decrease MCAM activity or disrupt endothelial cell adhesion in diseased tissue. Nucleic acids containing sequence derived from this gene, its complement, the gene product and active fragments thereof, and agonists and antagonists thereof may be used in any of the methods of therapy and diagnosis that are described above.

Claudin-4

Claudins are a family of transmembrane proteins that are involved in cell adhesion and the formation of tight junctions. They are small proteins of approximately 22 kDa and contain four potential transmembrane domains, each of approximately 20 amino acids. It has been suggested that claudins promote cell adhesion through homophilic interactions in which a claudin on the surface of one cell binds to an identical claudin on the surface of another cell, or alternatively heterophilic interactions, possibly with occludin, a protein known to be involved in tight junction formation and expressed in endothelial as well as epithelial cells (see WO 00/26360).

Claudin 4 mRNA levels were found to be higher in healthy tissue from patients C and D than in diseased tissue CR and DV and were completely absent from both healthy and diseased tissue of patients A and B. The cloned fragment identified as claudin 4 represents bases 479-692 of the human mRNA for claudin 4 (J03015) and subsequently residues 99-170 of claudin 4. Depressed levels of claudin 4 in endometriosis are suggestive of the disruption of tight junction formation and potentially increased permeability. Increased permeability may result in the release of compounds in the peritoneal cavity that cause some of the symptoms of endometriosis, such as pain. Interestingly, downregulation of the claudin family has been recently described in tumour microvessels of human glioblastoma multiforme and it has been suggested as responsible for the clinically severe symptoms of brain edema, due to the increase in microvascular permeability in human gliomas (Liebner S. et al., Acta Neuropathol., 2000, September; 100 (3): 323-331). To the best of our knowledge, the present study is the first report implicating tight junction dysregulation in endometriosis. The absence of claudin 4 transcript in patients A and B (Days 10 and 12) may be suggestive of an hormonal regulation of claudin 4 expression.

Depressed levels of this protein are therefore defined herein as being indicative of susceptibility to ovarian and recto-vaginal endometriosis. In order to treat or relieve the symptoms of these forms of endometriosis, methods should be used that effectively raise claudin 4 activity or decrease permeability by other methods in diseased tissue. Nucleic acids containing sequence derived from this gene, its complement, the gene product and active fragments thereof, and agonists and antagonists thereof may be used in any of the methods of therapy and diagnosis that are described above.

Other Proteins

A number of proteins with different functions has also been implicated in endometriosis. The transcription factor early growth response 1 (EGR1) has been found to be upregulated in the endometriotic lesions from patients A and B. No differences in expression levels were observed in patients D and C at Day 3 and 4 of the cycle indicating that EGR1 expression may be hormonally regulated, although the inventors do not wish to be bound to this theory.

In a similar manner RAD21, a DNA-double strand break repair gene, was differentially expressed in patients D (Days 3), K (Day 6), B (Day 12) and I (Day 24) with higher levels of RAD21 in the healthy endometrium. Depressed levels of RAD21 in endometriosis may suggest that the endometriotic lesions are deficient in their DNA repair mechanism.

Ribosomal protein S6 mRNA was also found to be upregulated in the healthy endometrium of patients A and B (Days 10 and 12) while RACK1 appeared to be differentially expressed between healthy endometrium and endometriosis in the early proliferative stage of the cycle (patients C and D) with no differences seen in the late proliferative, early secretory phase of the cycle.

The RNA editing enzyme adenosine deaminase, RNA-specific protein (ADAR), was found to be downregulated in endometriotic lesions during the mid-proliferative and early secretory phase of the menstrual cycle in patients N, K (Day 5 and 6 respectively) and P (Day 15).

CSRP-1

In a further embodiment of the invention, it has been discovered that the gene referred to as CSRP-1 (human cysteine and glycine rich protein) has a role in ovarian and recto-vaginal endometriosis.

Expression of this gene has been found to be raised in ovarian as well as rectovaginal endometriosis tissue through two different adaptor pools. Adaptor pool D identified a CSRP-1 fragment base pairs 15-365 (gb: M33146) being raised in diseased tissues from patients A and C (AV, CR) while adaptor pool C identified fragment 1-334 (gb: M33146) of CSRP-1 being overexpressed in patients A,C as well as B (AV, BV, CR). RT-PCR experiments confirmed the pattern of differential expression observed in DDRT-PCR. Further RT-PCR experiments with a larger number of patients has consistently shown elevated CSRP-1 mRNA in diseased tissues in 5 out of 8 patients with two patients having approximately equal amounts of CSRP-1 in both ectopic and eutopic endometrium.

The CSRP-1 fragments were discovered through two different adaptor pools (Pool C and D). Both fragments represent a very similar sequence (15-365 and 1-334 respectively). The fact that CSRP-1 fragments were isolated through two different adaptor pools provides confidence that CSRP-1 might represent a particularly important difference between healthy and diseased tissue.

Not much is known about the function of human cysteine and glycine rich protein. It is a 20.5 kDa protein containing four putative zinc fingers. CSRP-1 cDNA was identified and isolated from a human placental cDNA library on the basis of reproducible hybridisation at low stringency to a human prolactin probe. However its protein product lacks any significant evolutionary or functional relationship to human prolactin. The expression of the CSRP-1 gene is induced as a primary response to serum in quiescent Balb/c 3T3 cells and in human fibroblasts.

Investigations to date have been unable to identify any published records implicating CSRP-1 in endometrium function.

This aspect of the invention provides the CSRP-1 protein or an active fragment of this protein, or a gene encoding this protein or an active fragment thereof, for use as a pharmaceutical. Preferably, the CSRP-1 protein may be used in the manufacture of a medicament for the treatment or diagnosis of ovarian or recto-vaginal endometriosis.

As is discussed generally in some detail above, elevated levels of expression of the CSRP-1 gene are indicative of susceptibility to ovarian or recto-vaginal endometriosis. In order to treat endometriosis, methods should be used that effectively lower CSRP-1 activity in diseased tissue.

Transcobalamin II

The cellular uptake of cobalamin (vitamin B12) is mediated by transcobalamin II (TCII), a plasma protein that binds vitamin B12. The protein has been first identified as secreted by human umbilical vein endothelial (HUVE) cells.

Transcobalamin II is a non-glycosylated secretory protein of molecular mass 43 kDa, and together with its plasma membrane receptor (TC II-R), a heavily glycosylated protein with a monomeric molecular mass of 62 kDa, are essential components of plasma vitamin B 12 transport to all cells.

Transcobalamin II mRNA levels were found to be higher in diseased tissue in all four patients tested A. B, C and D. The cloned fragment identified as transcobalamin II represents bases 1022-1238 corresponding to residues 328-400 of the protein. Increased levels of transcobalamin II in endometriosis are suggestive of increased vitamin B12 transfer in the endometriotic lesion.

Increased levels of this protein are therefore defined herein as being indicative of susceptibility to endometriosis. In order to treat or relieve the symptoms of endometriosis, methods should be used that effectively decrease transcobalamin II levels in diseased tissue. Nucleic acids containing sequence derived from this gene, its complement, the gene product and active fragments thereof, and agonists and antagonists thereof may be used in any of the methods of therapy and diagnosis that are described above.

Finally, the endothelial cell surface protein podocalyxin have been found herein to be downregulated in endometriosis at patients K, B and I, Day 6, 12 and 15 respectively.

To the best of our knowledge, this is the first report suggesting that the mRNA of any of the molecules described above implicated in endometriosis. This aspect of the invention provides the use of the proteins described above or an active fragment of them, for use in the manufacture of a medicament for the treatment or diagnosis of endometriosis. The invention also provides the use of a nucleic acid molecule containing the EGR-1, human guanine nucleotide binding protein, podocalyxin, RAD21, ribosomal protein S6, ADAR gene sequence, nucleic acid molecules that hybridise to these gene sequences and nucleic acid molecules that encode gene products which are functionally equivalent to EGR-1, human guanine nucleotide binding protein, podocalyxin, RAD21, ribosomal protein S6, ADAR gene product, in the manufacture of a medicament for the treatment or diagnosis of endometriosis. Such molecules, or agonists and antagonists of these molecules, may also be used in methods of treatment of patients suspected of suffering from endometriosis.

1. A method of screening for a gene or gene product that is associated with endometriosis disease comprising comparing the pattern of gene expression in a diseased endometrium tissue from a patient suffering from endometriosis to the pattern of gene expression in healthy endometrium tissue from the same patient suffering from endometriosis, and selecting a gene whose level of expression differs between healthy and diseased tissues.

2. A method according to claim 1, wherein the pattern of gene expression in diseased and healthy endometrium tissues is assessed using a method of monitoring the differential expression of a gene.

3. A method according to claim 2, wherein said method of monitoring the differential expression of a gene comprises the indexing differential display reverse transcriptase polymerase chain reaction (DDRT-PCR).

4. A method for detecting or diagnosing endometriosis disease in a patient, comprising assessing the level of expression or biological activity of one or more genes or gene products identified by the method according to any one of claims 1-3 in tissue from said patient and comparing said level of expression or biological activity to a control level of expression or biological activity, wherein a level of expression that is different to said control level is indicative of endometriosis.

5. A method according to claim 3, wherein said control level of expression is the level of expression or biological activity of said gene or of said gene product in healthy endometrium tissue from said patient.

6. A method according to any one of claims 3-5, wherein said gene encodes a gene product selected from the group consisting of a protease or a protease inhibitor, a tumour suppressor protein, a protein of the immune system, a protein involved in an inflammatory response, an enzyme, a lipid binding protein, a transcription factor, a matrix or cell adhesion molecule.

7. A method according to any one of claims 4-7, wherein said gene encodes a gene product selected from the group consisting of cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF- 1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) and podocalyxin.

8. An array of at least two nucleic acid molecules, wherein each of said nucleic acid molecules either corresponds to the sequence of, is complementary to the sequence of, or hybridises specifically to a gene implicated in endometriosis as recited in claim 7.

9. An array according to claim 8, which contains nucleic acid molecules that either correspond to the sequence of, are complementary to the sequence of, or hybridise specifically to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 of the genes implicated in endometriosis as recited in claim 7.

10. An array according to claim 8 or claim 9, which contains a plurality of nucleic acid molecules of overlapping sequence, each nucleic acid molecule consisting of a portion of the consensus sequence of a gene implicated in endometriosis, or its complement, wherein a nucleic acid molecule is included on the array that corresponds to each of the four potential nucleotide variants at a number of nucleotide positions in the gene sequence, the remainder of the nucleic acid molecule corresponding to the wild type sequence of the gene.

11. An array according to claim 10, wherein a nucleic acid molecule is included on the array corresponding to each of the four potential nucleotide variants at every nucleotide position in the gene sequence.

12. An array according to any one of claims 8-11, wherein said nucleic acid molecules consist of between twelve and fifty nucleotides, more preferably, between fifteen and twenty-five nucleotides.

13. An array of antibodies, comprising at least two different antibody species, wherein each antibody species is immunospecific with a gene product of a gene implicated in endometriosis as recited in claim 7.

14. An array of polypeptides, comprising at least two polypeptide species, wherein each polypeptide species comprises a gene product of a gene implicated in endometriosis as recited in claim 7, or is a functional equivalent variant or fragment thereof.

15. An array according to any one of claims 8 to 14, wherein said genes or gene products belong to different functional categories selected from the group consisting of a protease or a protease inhibitor, a tumour suppressor protein, a protein of the immune system, a protein involved in an inflammatory response, an enzyme, a lipid binding protein, a transcription factor or a matrix or cell adhesion molecule.

16. An array according to any one of claims 8 to 15, wherein said genes or gene products belong to a single functional category selected from the group consisting of a protease or a protease inhibitor, a tumour suppressor protein, a protein of the immune system, a protein involved in an inflammatory response, an enzyme, a lipid binding protein, a transcription factor or a matrix or cell adhesion molecule.

17. A method according to any one of claims 4-7, comprising contacting a nucleic acid or protein sample from tissue of the patient with an array according to any one of claims 8-16 and detecting the binding of the polynucleotide or protein to the array.

18. A method according to any one of claims 4-7, comprising the steps of:
a) contacting a sample of nucleic acid from a tissue of a patient with a nucleic acid probe or nucleic acid array under stringent conditions that allow the formation of a hybrid complex between the patient nucleic acid and the probe or array;
b) contacting a control sample with said probe or array under the same conditions used in step a); and
c) detecting the presence of hybrid complexes in said samples;
wherein detection of levels of the hybrid complex in the patient sample that differ from levels of the hybrid complex in the control sample is indicative of endometriosis.

19. A method according to any one of claims 4-7, comprising:
a) contacting a sample of nucleic acid from tissue of the patient with a nucleic acid primer under stringent conditions that allow the formation of a hybrid complex between the gene and the probe;
b) contacting a control sample with said primer under the same conditions used in step a); and
c) amplifying the sampled nucleic acid using the primers; and
d) detecting the level of the amplified nucleic acid from both patient and control samples;
wherein detection of levels of the amplified nucleic acid in the patient sample that differ significantly from levels of the amplified nucleic acid in the control sample is indicative of endometriosis.

20. A method according to any one of claims 4-7 comprising:
a) obtaining a tissue sample from a patient being tested for endometriosis;
b) isolating nucleic acid encoding a gene according to claim 7 from said tissue sample; and
c) assessing the risk of a patient developing endometriosis on the basis of the presence or absence of a mutation in the nucleic acid sample which is associated with endometriosis.

21. The method of claim 20, further comprising amplifying the patient nucleic acid to form an amplified product and detecting the presence or absence of a mutation in the amplified product which is associated with endometriosis.

22. The method of claim 20 or claim 21, wherein said mutation is a point mutation.

23. The method of claim 22, wherein said point mutation is a missense mutation.

24. The method of any one of claims 18-23, wherein said patient nucleic acid that encodes said gene is cDNA reverse-transcribed from RNA.

25. The method of any one of claims 20-24, wherein the presence or absence of the mutation in the patient is detected by contacting said patient nucleic acid with a nucleic acid probe that hybridises to said patient nucleic acid under stringent conditions to form a hybrid double-stranded molecule, the hybrid double-stranded molecule having an unhybridised portion of the nucleic acid probe strand at any portion corresponding to a mutation associated with endometriosis; and
detecting the presence or absence of an unhybridised portion of the probe strand as an indication of the presence or absence of an endometriosis-associated mutation in the corresponding portion of the patient DNA strand.

26. The method according to claim 25, wherein said unhybridised portion of the probe strand is detected by contacting the hybrid double-stranded molecule with an agent capable of digesting an unhybridised portion of the hybrid double-stranded molecule.

27. A method according to any one of claims 4-7, comprising:
a) contacting a sample of tissue from the patient with an antibody that binds to a gene product as recited in claim 7 under conditions that allow for the formation of reaction complexes comprising the antibody and the gene product; and
b) contacting a control sample with said under the same conditions used in step a); and
c) detecting the formation of reaction complexes comprising the antibody and the gene product in said samples;
wherein detection of levels of reaction complex in the patient sample that differ significantly from levels of the reaction complex in the control sample is indicative of endometriosis.

28. The method of claim 27, wherein the antibody is bound to a solid phase support.

29. A method according to any one of claims 4-7, or 17-28, wherein the body sample or tissue is removed from said patient.

30. The method of any one of claims 4-7, or 17-29, wherein said tissue is endometrium tissue of a patient.

31. The method of claim 30, wherein said tissue is ectopic endometrium tissue of a patient.

32. The method of claim 31, wherein said ectopic endometrium tissue is in the vaginal, peritoneal, ovarian or rectovaginal area.

33. A method of monitoring the therapeutic effect of treatment of endometriosis disease in a patient, comprising monitoring over a period of time the level of expression or the biological activity of a gene or a gene product identified by the method according to any one of claims 1-3 in tissue from said patient, according to the method of any one of claims 4-7, or 15-32.

34. A method of treating endometriosis in a patient, said method comprising diagnosing the condition of the patient according to the method of any one of claims 4-7, or 15-33 and correlating the result of the diagnosis with an appropriate treatment for the patient.

35. A method of treating endometriosis in a patient comprising administering to the patient a compound that is effective to alter the expression or to regulate the activity of a gene or gene product identified by the method of any one of claims 1-3.

36. A method according to claim 35, wherein, for a gene or gene product whose level of expression is lower in diseased endometrium tissue as compared to the level of expression in healthy endometrium tissue, said compound comprises said gene, said gene product, an agonist of said gene or said gene product or a combination of one of more of said genes, gene products, or agonists.

37. A method according to claim 36, wherein said compound comprises a nucleic acid corresponding in sequence to the sequence of said gene, or to a portion of said gene, operatively linked to suitable control sequences that are effective to allow expression of the gene, or of the portion of the gene.

38. A method according to claim 36, wherein said compound comprises a therapeutically-effective amount of said gene product, optionally in conjunction with a pharmaceutical carrier and/or delivery system.

39. A method according to any one of claims 35-38, wherein said gene encodes, or said gene product is cystatin B; PAEP; pro-alpha-1 type III collagen; alpha-1 antitrypsin; IGFBP-3, claudin 4, prolyl 4 hydroxylase betapolypeptide, stromelysin 3, a protein in the wnt signalling pathway, sFRP4, alpha-2 type I collagen or type I procollagen C-terminal proteinase enhancer.

40. A method according to claim 35, wherein, for a gene or gene product whose level of expression is higher in diseased endometrium tissue as compared to the level of expression in healthy endometrium tissue, said compound comprises an antagonist of said gene or of said gene product.

41. A method according to claim 40, wherein said antagonist comprises an antisense nucleic acid molecule that specifically targets said gene.

42. A method according to claim 40, wherein said antagonist comprises a ribozyme molecule that specifically targets said gene.

43. A method according to claim 40, wherein said antagonist comprises an antibody that binds specifically to said gene product.

44. A method according to any one of claims 40-43, wherein said gene encodes, or said gene product is, cathepsin D; AEBP1; stromelysin-3; sFRP4; a protein in the wnt signalling pathway; gelsolin; Ferritin; complement component 3; immunoglobulin λ chain; vitamin D3 25 hydroxylase; CSRP1; transcobalamin II; dual specificity phosphatase 1; apolipoprotein E; Steroidogenic acute regulatory protein; an aspartic protease such as pepsinogen A, pepsinogen C, cathepsin E, renin; NACA; Prosaposin (SAP1); or Melanoma adhesion molecule.

45. A gene or gene product identified according to the method of any one of claims 1-3, for use as a pharmaceutical.

46. A gene or gene product according to claim 45, which is selected from the group consisting of cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) and podocalyxin.

47. Use of a gene or gene product identified by the method of any one of claims 1-3, or an agonist or antagonist of said gene or gene product, in the manufacture of a medicament for the diagnosis or treatment of endometriosis.

48. Use according to claim 47, wherein said gene encodes, or gene product is, cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type II collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) or podocalyxin.

49. A method for the identification of an agent that is effective in the treatment and/or diagnosis of endometriosis, comprising contacting a gene or gene product identified by the method according to any one of claims 1-3, such as recited in claim 7, with one or more compounds suspected of possessing binding affinity for said gene or gene product, and selecting as said agent, a compound that binds to said gene or gene product.

50. A kit useful for diagnosing endometriosis comprising a first container containing a nucleic acid probe that hybridises under stringent conditions with host nucleic acid which encodes a gene product identified according to the method of any one of claims 1-3; a second container containing primers useful for amplifying said host nucleic acid; and instructions for using the probe and primers for facilitating the diagnosis of endometriosis.

51. A kit according to claim 50, wherein said probe is an RNA or single-stranded DNA probe.

52. A kit of claim 50 or claim 51, further comprising a third container holding an agent for digesting unhybridised RNA.

53. A kit comprising one or more antibodies that bind to a gene product identified by the method of any one of claims 1-3; and a reagent useful for the detection of a binding reaction between said antibody and said gene product.

54. A kit according to any one of claims 50-53, wherein said gene encodes, or said gene product is cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) or podocalyxin.

55. A genetically-modified non-human animal that has been transformed to express higher, lower or absent levels of a gene or gene product identified by the method of any one of claims 1-3.

56. A non-human animal according to claim 55, that is a transgenic or knockout animal.

57. A non-human animal according to claim 55 or claim 56, wherein said gene encodes, or said gene product is gene product is cathepsin D, AEBP-1, stromelysin-3, cystatin B, protease inhibitor 1, sFRP4, gelsolin, IGFBP-3, dual specificity phosphatase 1, PAEP, immunoglobulin λ chain, ferritin, complement component 3, pro-alpha-1 type III collagen, proline 4-hydroxylase, alpha-2 type I collagen, claudin-4, melanoma adhesion protein, procollagen C-endopeptidase enhancer, nascent-polypeptide-associated complex alpha polypeptide, elongation factor 1 alpha (EF-1α), vitamin D3 25 hydroxylase, CSRP-1, steroidogenic acute regulatory protein, apolipoprotein E, transcobalamin II, prosaposin, early growth response 1 (EGR1), ribosomal protein S6, adenosine deaminase RNA-specific protein, RAD21, guanine nucleotide binding protein beta polypeptide 2-like 1 (RACK1) or podocalyxin.

58. A method for screening for an agent effective to treat endometriosis, by contacting a genetically-modified non-human animal according to any one of claims 55-57 with a candidate agent and determining the effect of the agent on the endometriosis disease of the animal.
